(19) United States
(12) Patent Application Publication
(10) Pub. No.: US 2007/0219144 A1
(43) Pub. Date: Sep. 20, 2007

(54) IMMUNOMODULATORY SACCHARIDE COMPOUNDS

(75) Inventor: Todd L. Lowary, Edmonton (CA)

Correspondence Address: FISH & RICHARDSON P.C., PO BOX 1022, MINNEAPOLIS, MN, 55440-1022, US

(73) Assignee: University of Alberta

(21) Appl. No.: 11/687,400

(22) Filed: Mar. 16, 2007

Related U.S. Application Data
(60) Provisional application No. 60/783,657, filed on Mar. 16, 2006.

(51) Int. Cl. A61K 31/70; C07H 15/00

(52) U.S. Cl. 514/24; 536/4.1

(57) ABSTRACT

Compounds comprising a saccharide molecule and a 5-deoxy-5-methylthio-xylofuranose (MTX) moiety or a 5-deoxy-5-methylsulfoxy-xylofuranose (MSX) moiety, and use of such compounds in methods for modulating inflammation and immune responses.

### CROSS-REFERENCE TO RELATED APPLICATIONS

This application claims priority from U.S. Provisional Application Ser. No. 60/783,657, filed Mar. 16, 2006.

### TECHNICAL FIELD

This invention relates to saccharide compounds having a 5-deoxy-5-methylthio-xylofuranose (MTX) or 5-deoxy-5-methylsulfoxy-xylofuranose (MSX) motif, and the use of such compounds to modulate immune responses and inflammation.

### BACKGROUND

Tuberculosis (TB) is the world's most lethal bacterial disease, killing more than 2 million people worldwide each year (Paolo and Nosanchuk (2004) Lancet Infect. Dis. 4:287-293; Kremer and Besra (2002) Expert Opin. Inv. Drugs 11:153-157; and Coker (2004) Trop. Med. Int. Health 9:25-40). Increased concern about the impact of this disease on world health has resulted from the emergence of multi-drug resistant strains of Mycobacterium tuberculosis (Nachega and Chaisson (2003) Clin. Infect. Dis. 36:S24-S30), the organism that causes the disease, and difficulties in treating individuals who have both TB and HIV (De Jong et al. (2004) Annu. Rev. Med. 55:283-301). A hallmark of TB and other mycobacterial diseases is the need for protracted treatments, typically involving multiple antibiotics that must be taken over several months (Bass et al. (1994) Am. J Respir. Crit. Care Med. 149:1359-1374). The need for this prolonged drug regimen is due to the unusual structure of the mycobacterial cell wall, which serves as a formidable barrier to the passage of antibiotics into the organism (Brennan (2003) Tuberculosis 83:91-97; and Lowary, “Mycobacterial Cell Wall Components.” In Glycoscience: Chemistry and Chemical Biology. Fraser-Reid, Tatsuta, and Thiem, Eds., Springer-Verlag: Berlin, 2001, pp 2005-2080). In addition to serving as a permeability barrier, the mycobacterial cell wall contains components that act as immunomodulatory molecules, enabling the organism to resist the immune system of the human host (Nigou et al. (2003) Biochemie 85:153-166; and Briken et al. (2004) Mol. Microbiol. 53: 391403).

### SUMMARY

The present document is based in part on the discovery that 5-deoxy-5-methylthio-xylofuranose (MTX) or 5-deoxy-5-methylsulfoxy-xylofuranose (MSX) residues present in glycolipids contained within the mycobacterial cell wall may play a role in the immune response arising from mycobacterial infection. Through the combined use of chemical synthesis and NMR spectroscopy, the inventor established that the MTX/MSX residues in these glycoconjugates are of the D-configuration and are linked α-(1→4) to a mannopyranose residue in the mannan portion of the glycan. Conformational analysis of the MTX/MSX residue using NMR spectroscopy showed differences in ring conformation and as well as in the rotamer populations about the C-4-C-5 bond, as compared to the parent compound, methyl α-D-xylofuranoside. Disaccharides based on this motif were synthesized, tested in cytokine induction assays, and shown to inhibit production of tumor necrosis factor-α (TNF-α) and interleukin-12 (IL-12) in response to treatment with a preparation of interferon-γ (IFN-γ) and Staphylococcus aureus Cowan strain (IFN/SAC-γ). Thus, the MTX/MSX class of compounds may be useful in modulating immune responses, treating inflammation and immune disorders such as rheumatoid arthritis, and modulating cytokine levels.

In one aspect, this document features a saccharide compound comprising a 5-deoxy-5-methylthio-xylofuranose (MTX) or 5-deoxy-5-methylsulfoxy-xylofuranose (MSX) moiety, or a derivative thereof The MTX or MSX moiety or derivative thereof can be in the D-configuration. The saccharide can be a monosaccharide or a disaccharide. The compound can be selected from Formulas 1-6: 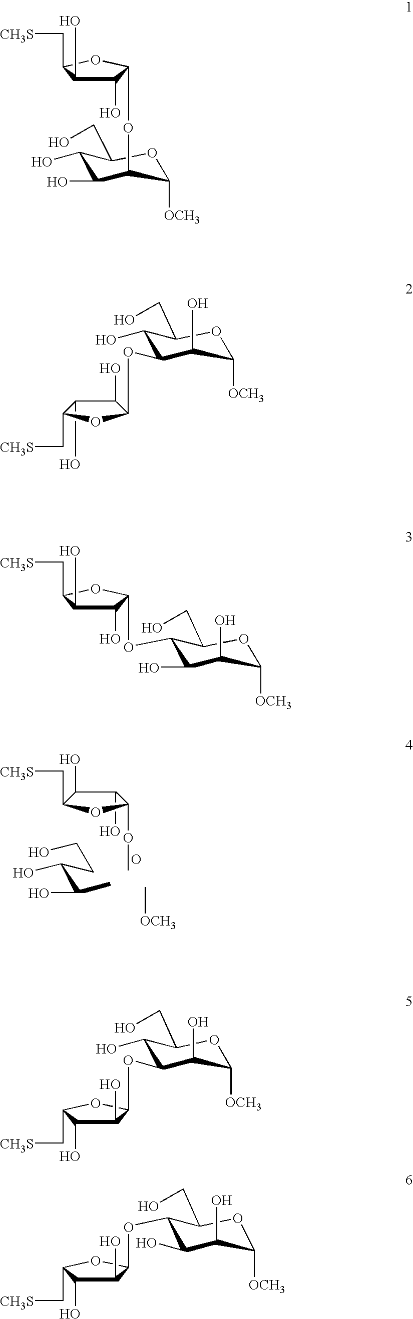
The compound can be the oxidized form of any one of compounds 1-6. The compound can be Formula 34:

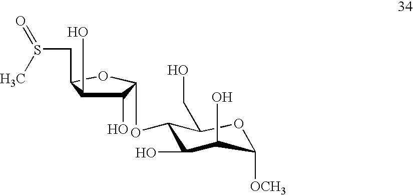

In another aspect, this document features a compound comprising a derivative of a MTX or MSX moiety, wherein the compound is a tosylated thioglycoside. The derivative of a MTX or MSX moiety can be selected from Formula 7 and Formula 8:

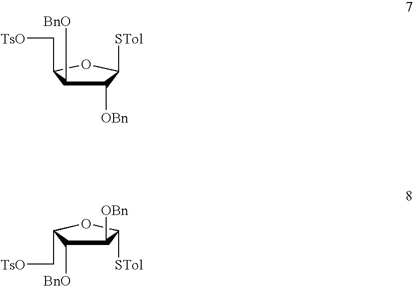

This document also features a composition comprising a saccharide compound as described herein. The composition can further comprise a pharmaceutically acceptable carrier.

In another aspect, this document features a method for modulating an immune response in a mammal, comprising administering to a mammal in need thereof a saccharide compound or a composition as described herein.

In still another aspect, this document features a method for treating an inflammatory disorder in a mammal, comprising administering to a mammal diagnosed with an inflammatory disorder a saccharide compound or a composition as described herein.

In another aspect, this document features a method for treating an autoimmune disorder in a mammal, comprising administering to a mammal diagnosed with an autoimmune disorder (e.g., rheumatoid arthritis) a saccharide compound or a composition as described herein.

In yet another aspect, this document features a method for modulating cytokine, lymphokine, or chemokine levels in a mammal, comprising administering to the mammal a saccharide compound or a composition as described herein. The cytokine, lymphokine, or chemokine can be TNF-α or IL-12.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1 is a schematic representation of the major structural domains in mycobacterial LAM.

FIG. 2 is a chart showing the disaccharides 1-6, which were synthesized to investigate the conformation of MTX/MSX residues.

FIG. 3 is a chart showing the five building blocks (7-11) used to assemble disaccharides 1-6.

FIG. 4 depicts Schemes 1 and 2 for synthesis of 7 and 8, respectively.

FIG. 5 depicts Scheme 3, for synthesis of disaccharides containing the D-enantiomer of MTX (1-3).

FIG. 6 depicts Scheme 4, for synthesis of disaccharides containing an L-MTX residue (4-6).

FIG. 7 depicts Scheme 5, for oxidizing 3 into the corresponding diastereomeric mixture of sulfoxides.

FIG. 8 is a chart showing 35 and 36.

FIG. 9 depicts a pseudorotational wheel for a D-aldofuranose ring.

FIG. 10 illustrates gg, gt and tg rotamers about the C-4C-5 bond.

FIGS. 11A and 11B are graphs showing the effect of various LAM derivatives on production of TNF-α (FIG. 11A) and IL-12p70 (FIG. 11B).

FIG. 12 is a graph showing the effect of IFN-γ/SAC on TNF-α production following pre-incubation with various LAM derivatives. Man=ManLAM; Ara=AraLAM.

FIG. 13 is a graph showing the effect of IFN-γ/SAC on IL-12 production following pre-incubation with various LAM derivatives. Man=ManLAM; Ara=AraLAM.

### DETAILED DESCRIPTION

Among the many components that make up the mycobacterial cell wall is a glycolipid, lipoarabinomannan (LAM), which is a major antigen. Mycobacterial LAM has been implicated in a large number of important immunological events (Brennan, supra; and Lowary, supra). For example, in the case of M. tuberculosis, it is believed that this polysaccharide is of critical importance in allowing the organism to survive in host macrophages.

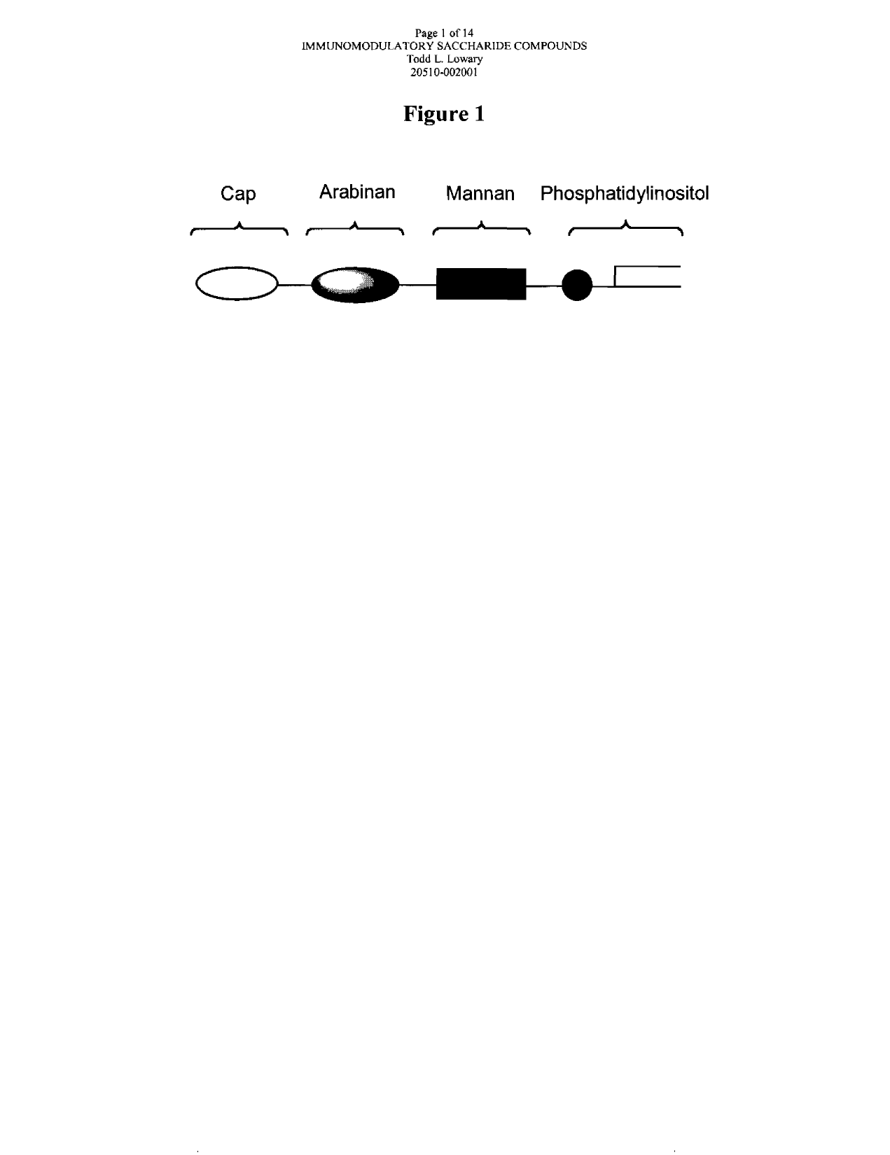

The fine structure of mycobacterial LAM is shown in FIG. 1. At its core is a phosphatidylinositol moiety to which is attached a mannan consisting of α-(1→6) and α-(1→2)-linked mannopyranose residues. An arabinan domain, composed of α-(1→5), α-(1→3), and β-(1→2)-linked arabinofuranose residues, is attached to the mannan chain. This arabinan is often further functionalized at its non-reducing terminus with “capping” motifs of varying structure. In M. tuberculosis, M. bovis and M. avium, the predominant capping motifs are small α-(1→2)-linked mannopyranosyl oligosaccharides, which, when present, give rise to a LAM variant termed ManLAM. (Nigou et al. (1997) J. Biol. Chem. 272:23094-23103; and Khoo et al. (2001) J. Biol. Chem. 276:3863-3871). In contrast, in M. smegmatis, the marmose caps are replaced with inositol phosphate moieties, resulting in a glycolipid called PILAM (Khoo et al. (1995) J. Biol. Chem. 270:1238012389). At least some of the immunomodulatory role of LAM has been ascribed to these capping motifs.

The structures of LAM molecules from a range of mycobacteria and other actinomycetes have been reported. See, for example, Guérardel et al. (2002) J. Biol. Chem. 277:30635-30648; Torrelles et al. (2004) J. Biol. Chem. 279:41227-41239; Gibson et al. (2003) Biochem. J. 372:821-829; Gibson et al. (2004) J. Biol. Chem. 279:22973-22982; Gibson et al. (2003) Microbiol. 149:1437-1445; Garton et al. (2002) J. Biol. Chem. 277:31722-31733; Gilleron et al. (2005) J. Bacteriol. 187:854-861; Sutcliffe (2000) Antonie Van Leeuwenhoek 78:195-201; Flaherty and Sutcliffe (1999) Syst. Appl. Microbiol. 22:530-533; Flaherty et al. (1996) Zentralbl. Bakteriol. 285:11-19; and Gibson et al. (2005) J. Biol. Chem. 280:28347-28356. LAM from a number of M. tuberculosis strains contain a 5-deoxy-5-methylthio-pentose residue. This substituent has been identified in both laboratory strains (H37Rv and H37Ra), as well as clinical isolates (CSU20) and MT 103) of M. tuberculosis (Treumann et al. (2002) J. Mol. Biol. 316:89-100; and Ludwiczak et al. (2002) Microbiol. 148:3029-3037).

The 5-deoxy-5-methylthio-pentose residue is linked to the mannopyranose capping residues. This motif is a 5-deoxy-5-methylthio-a-xylofuranose (MTX) residue (Turnbull et al. (2004) Angew. Chem. Intl. Ed. 43:3918-3922). The MTX moiety also has been found in M. kansasii, where it is attached not to the mannopyranose capping residues, but rather to the mannan core (Guérardel et al. (2003) J. Biol. Chem. 278:36637-36651). In addition to MTX, the corresponding sulfoxide, 5-deoxy-5-methylsulfoxy-xylofuranose (MSX), also is present in these polysaccharides.

The present document is based in part on the discoveries that the MTX/MSX residues in LAM may play a role in the immune response arising from mycobacterial infection, and that MTX- and MSX-containing compounds may be useful to modulate immune responses, treat autoimmune and inflammatory disorders, and modulate lymphokine and cytokine levels. As is described herein, the inventor synthesized a panel of MTX and MSX-containing disaccharides, which were used in NMR studies to demonstrate that these monosaccharides have the D configuration and are attached to LAM via an α-(1→4)-linkage to a mannopyranose residue. Also described herein are experiments to elucidate the conformation of the MTX/MSX substituent and to test the ability of the synthesized disaccharides to induce or suppress cytokine production.

Saccharide Compounds

This document provides saccharide compounds having a MTX or MSX moiety, or a derivative of a MTX or MSX moiety. As used herein, the term “saccharide compound” refers to a compound comprising at least one monosaccharide unit. A monosaccharide is a simple sugar that cannot be hydrolyzed to smaller units. With a few exceptions, monosaccharides have the chemical formula (CH₂O)ₙ₊ₘ and the chemical structure H(CHOH)ₙC═O(CHOH)ₘH. Naturally occurring saccharides typically range in size from trioses (n=3) to heptoses (n=7). Monosaccharides can be classified according to their molecular configuration at the stereogenic carbon furthest from the carbonyl (C═O) group. As described herein, naturally-occurring MTX and MSX moieties have the D configuration.

The MTX- and MSX-containing saccharide compounds provided herein include at least one MTX or MSX moiety, or a derivative thereof. The MTX and MSX moieties are themselves monosaccharides. Thus, in some embodiments, a MTX- or MSX-containing saccharide compound can be a MTX or MSX molecule, or a derivative thereof. In other embodiments, an MTX or MSX motif can be linked to another molecule (e.g., another saccharide such as a monosaccharide, a disaccharide, or a higher order polysaccharide). As depicted in Formulas 1-6 herein, for example, a MTX moiety can be linked to a mannopyranoside shown in Formulas 9-11 to form a disaccharide.

A MTX or MSX derivative can be, for example, a salt of a MTX or MSX moiety, or a protected MTX or MSX molecule. Salts include, for example, salts formed with cations (e.g., sodium, potassium, calcium, or polyamines such as spermine); acid addition salts formed with inorganic acids (e.g., hydrochloric acid, hydrobromic acid, sulfuric acid, phosphoric acid, or nitric acid); and salts formed with organic acids (e.g., acetic acid, citric acid, oxalic acid, palmitic acid, or fumaric acid). In some embodiments, a MTX or MSX derivative can include one or more protecting groups. For example, a MTX or MSX molecule can be modified at one or more positions with a tosyl group, a benzyl group, an acetyl group, a benzoyl group, a tolyl group, an acetyl group, an allyl group, an aryl group, a methyl group, a phenyl group, a trityl group, a methoxybenzyl group, a toluenesulfonic acid group, and/or a tricholorethoxycarbonyl group. In some cases, a MTX or MSX moiety can be derivatized with tosyl and benzyl groups (e.g., as in compound 7 and 8 described herein). Other suitable derivatives of a saccharide compound include esters, enol ethers, enol esters, acetals, ketals, orthoesters, hemiacetals, hemiketals, acids, bases, solvates, hydrates or prodrugs thereof. MTX and MSX derivatives can be readily prepared by those of skill in the art using known methods for such derivatization.

The saccharide compounds described herein can be obtained using any suitable methods, including those that are known in the art. As described herein, for example, saccharide compounds can be prepared using organic synthesis methods. Alternatively, saccharide compounds can be obtained by extraction from a natural source (e.g., from isolated Mycobacterium cells).

In some embodiments, a saccharide compound can be “purified” or “isolated.” As used herein, the terms “purified” and “isolated” refer to a saccharide compound that either has no naturally occurring counterpart, has been chemically synthesized and is thus substantially uncontaminated by other molecules (e.g., synthesis intermediates), or has been separated or purified from other cellular components by which it is naturally accompanied.

Compositions

This document also provides compositions comprising the MTX- and MSX-containing saccharide compounds described herein. A “pharmaceutical composition” comprises a disclosed saccharide compound in conjunction with an acceptable pharmaceutical carrier as part of a pharmaceutical composition for administration to a subject (e.g., a mamma] such as a mouse, rat, horse, sheep, pig, cow, dog, cat, rabbit, non-human primate, or human). Formulation of the compound to be administered will vary according to the route of administration selected (e.g., oral, intravenous (i.v.), parenteral, or topical administration, using a solution, emulsion, tablet, capsule, cream, ointment, and the like). Suitable pharmaceutical carriers may contain inert ingredients that do not interact with the compound. Standard pharmaceutical formulation techniques can be employed, such as those described in Remington's Pharmaceutical Sciences, Mack Publishing Company, Easton, Pa.

A “pharmaceutically acceptable carrier” (also referred to herein as an “excipient”) is a pharmaceutically acceptable solvent, suspending agent, or any other pharmacologically inert vehicle for delivering one or more MTX- or MSX-containing saccharide compounds to a subject. Pharmaceutically acceptable carriers can be liquid or solid, and can be selected with the planned manner of administration in mind so as to provide for the desired bulk, consistency, and other pertinent transport and chemical properties, when combined with one or more saccharide compounds and any other components of a given pharmaceutical composition. Pharmaceutically acceptable carriers include, without limitation, water, saline solution, binding agents (e.g., polyvinylpyrrolidone or hydroxypropyl methylcellulose), fillers (e.g., lactose and other sugars, gelatin, or calcium sulfate), lubricants (e.g., starch, polyethylene glycol, or sodium acetate), disintegrates (e.g., starch or sodium starch glycolate), and wetting agents (e.g., sodium lauryl sulfate).

The compositions provided herein can be administered by a number of methods, depending upon whether local or systemic treatment is desired and upon the area to be treated. Administration can be, for example, topical (e.g., transdermal, sublingual, ophthalmic, or intranasal); pulmonary (e.g., by inhalation or insufflation of powders or aerosols); oral; or parenteral (e.g., by subcutaneous, intrathecal, intraventricular, intramuscular, or intraperitoneal injection, or by intravenous drip). Administration can be rapid (e.g., by injection) or can occur over a period of time (e.g., by slow infusion or administration of slow release formulations). For treating tissues in the central nervous system, saccharide compounds can be administered by injection or infusion into the cerebrospinal fluid, preferably with one or more agents capable of promoting penetration of the compounds across the blood-brain barrier.

Formulations for topical administration of saccharide compounds include, for example, sterile and non-sterile aqueous solutions, non-aqueous solutions in common solvents such as alcohols, or solutions in liquid or solid oil bases. Such solutions also can contain buffers, diluents and other suitable additives. Pharmaceutical compositions and formulations for topical administration can include transdermal patches, ointments, lotions, creams, gels, drops, suppositories, sprays, liquids, and powders. Nasal sprays are particularly useful, and can be administered by, for example, a nebulizer or another nasal spray device. Administration by an inhaler also is particularly useful. Conventional pharmaceutical carriers, aqueous, powder or oily bases, thickeners and the like may be necessary or desirable.

Compositions and formulations for oral administration include, for example, powders or granules, suspensions or solutions in water or non-aqueous media, capsules, sachets, or tablets. Such compositions also can incorporate thickeners, flavoring agents, diluents, emulsifiers, dispersing aids, or binders.

Compositions and formulations for parenteral, intrathecal or intraventricular administration can include sterile aqueous solutions, which also can contain buffers, diluents and other suitable additives (e.g., penetration enhancers, carrier compounds and other pharmaceutically acceptable carriers).

Pharmaceutical compositions include, but are not limited to, solutions, emulsions, aqueous suspensions, and liposome-containing formulations. These compositions can be generated from a variety of components that include, for example, preformed liquids, self-emulsifying solids and self-emulsifying semisolids. Emulsions are often biphasic systems comprising of two immiscible liquid phases intimately mixed and dispersed with each other; in general, emulsions are either of the water-in-oil (w/o) or oil-in-water (o/w) variety. Emulsion formulations have been widely used for oral delivery of therapeutics due to their ease of formulation and efficacy of solubilization, absorption, and bioavailability.

Liposomes are vesicles that have a membrane formed from a lipophilic material and an aqueous interior that can contain the composition to be delivered. Liposomes can be particularly useful due to their specificity and the duration of action they offer from the standpoint of drug delivery. Liposome compositions can be formed, for example, from phosphatidylcholine, dimyristoyl phosphatidylcholine, dipalmitoyl phosphatidylcholine, dimyristoyl phosphatidylglycerol, or diolcoyl phosphatidylethanolamine. Numerous lipophilic agents are commercially available, including LIPOFECTIN® (Invitrogen/Life Technologies, Carlsbad, Calif.) and EFFECTENE™ (Qiagen, Valencia, Calif.).

The saccharide compounds provided herein can further encompass any pharmaceutically acceptable salts, esters, or salts of such esters, or any other compound which, upon administration to a mammal such as a human, is capable of providing (directly or indirectly) the biologically active metabolite or residue thereof. Accordingly, for example, this document provides pharmaceutically acceptable salts of saccharides such as MTX and MSX, prodrugs and pharmaceutically acceptable salts of such prodrugs, and other bioequivalents. The term is “prodrug” indicates a therapeutic agent that is prepared in an inactive form and is converted to an active form (i.e., drug) within the body or cells thereof by the action of endogenous enzymes or other chemicals and/or conditions. The term “pharmaceutically acceptable salts” refers to physiologically and pharmaceutically acceptable salts of the saccharide compounds provided herein (i.e., salts that retain the desired biological activity of the parent saccharide molecule without imparting undesired toxicological effects). Examples of pharmaceutically acceptable salts include, but are not limited to, salts formed with cations (e.g., sodium, potassium, calcium, lithium, magnesium, barium, zinc, or polyamines such as spermine), acid addition salts formed with inorganic acids (e.g., hydrochloric acid, hydrobromic acid, sulfuric acid, phosphoric acid, or nitric acid), and salts formed with organic acids (e.g., acetic acid, citric acid, oxalic acid, palmitic acid, or fumaric acid). Pharmaceutically acceptable salts also include amine salts, salts of mineral acids (e.g., hydrochlorides, hydrobromides, hydroiodides and sulfates), and salts of organic acids (e.g., acetates, trifluoroacetates, maleates, oxalates, lactates, malates, tartrates, citrates, benzoates, salicylates, ascorbates, succinates, butyrates, valerates and fumarates). In some embodiments, a composition can be a solvate or hydrate of a saccharide compound. Pharmaceutically acceptable solvates and hydrates are complexes of a compound with one or more (e.g., 1 to about 100, 1 to about 10, or one to about 2, 3, or 4) solvent or water molecules.

Pharmaceutical compositions containing the saccharide compounds provided herein also can incorporate penetration enhancers that promote the efficient delivery of saccharide compounds as described herein to the skin of animals. Penetration enhancers can enhance the diffusion of both lipophilic and non-lipophilic drugs across cell membranes. Penetration enhancers can be classified as belonging to one of five broad categories, i.e., surfactants (e.g., sodium lauryl sulfate, polyoxyethylene-9-lauryl ether and polyoxyethylene-20-cetyl ether); fatty acids (e.g., oleic acid, lauric acid, myristic acid, palmitic acid, and stearic acid); bile salts (e.g., cholic acid, dehydrocholic acid, and deoxycholic acid); chelating agents (e.g., disodium ethylenediaminetetraacetate, citric acid, and salicylates); and non-chelating non-surfactants (e.g., unsaturated cyclic ureas).

In some embodiments, a composition can contain (a) one or more saccharide compounds and (b) one or more other agents that function by a different mechanism. For example, anti-inflammatory drugs, including but not limited to nonsteroidal anti-inflammatory drugs and corticosteroids, and antiviral drugs, including but not limited to ribivirin, vidarabine, acyclovir and ganciclovir, can be included in the compositions provided herein. Other non-saccharide agents also are within the scope of this document. Such combined compounds can be used together or sequentially.

Compositions additionally can contain other adjunct components conventionally found in pharmaceutical compositions. Thus, the compositions also can include compatible, pharmaceutically active materials such as, for example, antipruritics, astringents, local anesthetics or anti-inflammatory agents, or additional materials useful in physically formulating various dosage forms of the compositions of the present invention, such as dyes, flavoring agents, preservatives, antioxidants, opacifiers, thickening agents and stabilizers. Furthermore, the composition can be mixed with auxiliary agents, e.g., lubricants, preservatives, stabilizers, wetting agents, emulsifiers, salts for influencing osmotic pressure, buffers, colorings, flavorings, and aromatic substances. When added, however, such materials should not unduly interfere with the biological activities of the saccharide components within the compositions. The formulations can be sterilized if desired.

The pharmaceutical formulations of the compositions provided herein, which can be presented conveniently in unit dosage form, can be prepared according to conventional techniques well known in the pharmaceutical industry. Such techniques include the step of bringing into association the active ingredients (e.g., the MTX- or MSX-containing saccharide compound) with the desired pharmaceutical carrier(s) or excipient(s). Typically, the formulations can be prepared by uniformly and bringing the active ingredients into intimate association with liquid carriers or finely divided solid carriers or both, and then, if necessary, shaping the product. Formulations can be sterilized if desired, provided that the method of sterilization does not interfere with the effectiveness of the saccharide compound contained in the formulation.

The compositions provided herein can be formulated into any of many possible dosage forms such as, but not limited to, tablets, capsules, liquid syrups, soft gels, suppositories, and enemas. The compositions also can be formulated as suspensions in aqueous, non-aqueous or mixed media. Aqueous suspensions further can contain substances that increase the viscosity of the suspension including, for example, sodium carboxymethylcellulose, sorbitol, and/or dextran. Suspensions also can contain stabilizers.

The MTX- and MSX-containing saccharide compounds provided herein can be used in the manufacture of a medicament (i.e., a composition) for treating conditions such as inflammatory disorders and autoimmune disorders (e.g., disorders that arise from abnormal Fc-mediated immune complex formation). Compositions typically contain one or more saccharide compounds as described herein. A MTX- or MSX-containing saccharide compound, for example, can be in a pharmaceutically acceptable carrier or diluent, and can be administered in amounts and for periods of time that will vary depending upon the nature of the particular disease, its severity, and the subject's overall condition. Typically, the compound is administered in an inhibitory amount (e.g., in an amount that is effective for reducing inflammation or inhibiting production of immune complexes in the cells or tissues contacted by the compound). The saccharide compounds and methods of the invention also can be used prophylactically, for example, to minimize immunoreactivity in a subject at risk for abnormal or over-production of immune complexes (e.g., a transplant recipient).

MTX- or MSX-containing saccharide compounds can be combined with packaging material and sold as kits for reducing inflammation, decreasing immune complex formation, or modulating immune responses, for example. Components and methods for producing articles of manufacture are well known. The articles of manufacture may combine one or more of the compounds set forth herein. In addition, the article of manufacture further may include, for example, buffers or other control reagents for reducing or monitoring reduced immune complex formation and inflammation. Instructions describing how the compounds and compositions are effective for reducing inflammation or modulating immune responses, for example, can be included in such kits.

Methods

This document provides methods using the compounds and compositions described herein to modulate immune responses and to treat conditions such as inflammatory disorders and autoimmune disorders in a subject (e.g., a human or another mammal). According to the methods provided herein, one or more MTX- and MSX-containing saccharide compounds, or a composition containing one or more of such compounds, can be administered to a subject having, for example, an inflammatory or autoimmune disorder. Saccharide compounds also can be used to modulate the level of cytokines, chemokines, and lymphokines (including, without limitation, TNF-α, IL-12, interferon-γ (IFN-γ), IL-1, IL-2, IL-4, IL-10, IL-13, IL-18, IFN-α, granulocyte macrophage-colony stimulating factor (GM-CSF) and transforming growth factor-β (TGF-β)).

The ability of a saccharide compound as described herein to modulate (e.g., reduce) an immune response or to treat (e.g., reduce the symptoms of) an autoimmune disorder can be assessed by, for example, measuring immune complex levels in a subject before and after treatment. A number of methods can be used to measure immune complex levels in tissues or biological samples, including methods that are known in the art. If the subject is a research animal, for example, immune complex levels in the joints can be assessed by immunostaining following euthanasia. The effectiveness of saccharide compound also can be assessed by direct methods such as measuring the level of circulating immune complexes in serum samples. Alternatively, indirect methods can be used to evaluate the effectiveness of saccharide compounds to treat autoimmune disorders and inflammatory disorders in live subjects by, for example, assessing a reduction in one or more symptoms of the disorder. For example, reduced immune complex formation can be inferred from reduced pain in rheumatoid arthritis patients. Animal models also can be used to study the development of and relief from conditions such as rheumatoid arthritis and other conditions including, without limitation, those set forth herein. The ability of a saccharide compound as described herein to modulate (e.g., increase or reduce) the level of a cytokine or lymphokine can be determined using any suitable method to evaluate the level of the cytokine or lymphokine, including those described in Example 5 herein, for example.

Methods for formulating and subsequently administering therapeutic compositions are well known to those skilled in the art. Dosing generally is dependent on the severity and responsiveness of the disease state to be treated, with the course of treatment lasting from several days to several months, or until a cure is effected or a diminution of the disease state is achieved. Persons of ordinary skill in the art routinely determine optimum dosages, dosing methodologies and repetition rates. Optimum dosages can vary depending on the relative potency of individual polypeptides, and can generally be estimated based on EC₅₀ found to be effective in in vitro and in vivo animal models. Typically, dosage is from 0.01 μg to 100 g per kg of body weight, and may be given once or more daily, biweekly, weekly, monthly, or even less often. Following successful treatment, it may be desirable to have the patient undergo maintenance therapy to prevent the recurrence of the disease state.

The methods provided herein can be used to treat a subject having, for example, rheumatoid arthritis (RA), systemic lupus erythematosus (SLE), lupus nephritis, autoimmune glomerulonephritis, atherosclerosis, multiple sclerosis (MS), Parkinson's disease, Crohn's disease, psoriasis, or ankylosing spondylitis (AS). The methods also can be used to modulate inflammation or rejection in a transplant recipient. These conditions are described in the subsections below. The methods also can include steps for identifying a subject in need of such treatment and/or monitoring treated subjects for a reduction in symptoms, for example.

Rheumatoid Arthritis (RA)—RA is characterized by chronic joint inflammation that eventually leads to irreversible cartilage destruction. In RA, abnormal IgG antibodies are produced by lymphocytes in the synovial membranes. These abnormal IgG antibodies then act as antigens. Other IgG and IgM antibodies, termed Rheumatoid Factors (RF), are present in sera and synovia and subsequently react with these abnormal IgG antibody/antigens to produce immune complexes. Immune complexes containing RF are abundant in synovial tissue of patients with RA. The presence of RF is associated with systemic symptoms, joint erosion, and poor prognosis, although the exact role of MF in RA remains to be fully elucidated.

Systemic lupus erythematosus (SLE) and lupus nephritis—SLE is a chronic autoimmune disease with many manifestations. The production of autoantibodies leads to immune complex formation and subsequent deposition in many tissues (e.g., glomeruli, skin, lungs, synovium, and mesothelium), leading to the manifestations of the disease. Renal disease is common with SLE because the immune complexes often are deposited in the renal glomeruli.

Lupus nephritis is an inflammation of the kidney that is caused by SLE-related glomerular deposition of immune complexes and FcγR (see, e.g., Clynes et al. (1998) Science 279:1052-1054). Proteinuria can be observed concomitant with the serological appearance of antibodies to DNA and histones, as well as immune complexes of the IgG1, IgG2a, and IgG2b subclasses. The median survival is 6 months, and mortality results from renal failure. B cells and autoantibodies are thought to play essential roles in disease development, and agents that interfere with autoantibody production have been shown to attenuate the disease.

Autoimmune glomerulonephritis—Autoimmune glomerulonephritis is related to lupus nephritis, and is due to a T cell dependent polyclonal B cell activation that is responsible for production of antibodies against self components (e.g., GBM, immunoglobulins, DNA, myeloperoxydase) and non self components (e.g., sheep red blood cells and trinitrophenol). Increased serum IgE concentration is the hallmark of this disease.

Atherosclerosis—Atherosclerotic lesions are thought to be largely of an inflammatory nature. Recent studies have focused on the inflammatory component of atherosclerosis, attempting to highlight the differences between stable and unstable coronary plaques. An increasing body of evidence supports the hypothesis that atherosclerosis shares many similarities with other inflammatory/autoimmune diseases. For example, similarities in the inflammatory/immunologic response have been observed in atherosclerosis, unstable angina, and RA, the prototype of autoimmune disease (Pasceri and Yeh (1999) Circulation 100:2124-2126).

Multiple sclerosis (MS)—MS is an autoimmune disease that attacks the insulating myelin sheath that surrounds neurons. This compromises conduction of nerve signals between the body and brain. Symptoms can be mild or severe, short or long in duration, and may include blurred vision, blindness, dizziness, numbness, muscle weakness, lack of coordination and balance, speech impediments, fatigue, tremors, sexual dysfunction, and bowel and bladder problems. Although many people have partial or complete remissions, symptoms for some progressively worsen with few or no remissions. Patients with MS may have ongoing systemic virus production with resultant immune complex formation. In addition, MS patients often have serum complexes containing brain-reactive components (Coyle and Procyk-Dougherty (1984) Ann. Neurol. 16:660-667).

Parkinson's disease (PD)—The clinical symptoms of PD result from the death of dopaminergic neurons in the substantia nigra section of the brain. An over responsive immune system may play a role in perpetuating PD by producing cytokines (e.g., interleukin-1 and tumor necrosis factor) in response to the initial damage, which can further injure cells in the brain. Furthermore, immunoglobulins from PD individuals have been shown to contribute to the pathogenesis of substantia nigra cells (Chen et al. (1998) Arch. Neurol. 55.1075-1080).

Crohn's disease—Crohn's disease results in chronic inflammation of the gastrointestinal tract, typically the small intestine. Crohn's disease can cause mild to severe abdominal pain, diarrhea, fever and weight loss. It is thought that the intestinal immune system of Crohn's patients over-reacts to viral or bacterial agents and initiates ongoing, uncontrolled inflammation of the intestine.

Psoriasis—Psoriasis is a chronic inflammatory skin condition. Those who develop psoriasis may get a related form of arthritis called “psoriatic arthritis,” which causes inflammation of the joints. The symptoms vary with the type of psoriasis, and can include patches of raised, reddish skin covered by silvery-white scale, red spots on the skin, white pustules surrounded by red skin, smooth red lesions in skin folds, or widespread redness, severe itching, and pain. In each type of psoriasis, the skin typically itches, and may crack and bleed.

Ankylosing Spondylitis (AS)—Ankylosing spondylitis is a form of chronic inflammation of the spine and the sacroiliac joints, which causes pain and stiffness in and around the spine. Over time, chronic spinal inflammation (spondylitis) can lead to a complete fusion of the vertebrae (ankylosis), leading to loss of mobility of the spine. AS also is a systemic rheumatic disease that can affect other tissues throughout the body. Accordingly, AS can cause inflammation in or injury to other joints away from the spine, as well as other organs, such as the eyes, heart, lungs, and kidneys.

Graft rejection following transplantation—Graft rejection typically results from the cumulative effects of both cell-mediated and humoral immune attacks on the grafted tissue. Solid organ (tissue) transplantation includes, for example, transfer of kidney, heart, lungs, liver, pancreas, skin, cornea, and bone. Bone marrow transplantation is employed in the treatment of conditions such as immunodeficiency disease, aplastic anemia, leukemia, lymphoma, and genetic disorders of hematopoiesis.

The invention will be further described in the following examples, which do not limit the scope of the invention described in the claims.

### EXAMPLES

### Example 1

### Synthesis of Targets

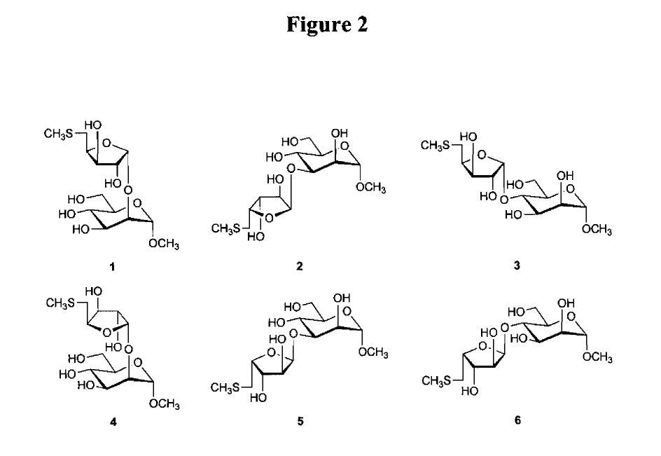

Approach: Through NMR spectroscopic investigations on ¹³C-labeled LAM from M. tuberculosis H37Ra, it was proposed that the MTX residue is linked to the mannopyranose capping units (Treumann et al., supra). As part of these studies, an HMBC experiment revealed a correlation between the anomeric hydrogen resonance of the MTX residue and a signal at 77.0 ppm in the ¹³C NMR spectrum. Similarly, the anomeric carbon resonance of the MTX residue correlated with a signal at 3.77 ppm in the ¹H NMR spectrum. These data suggested that the linkage of the MTX to the mannose caps is via a secondary hydroxyl group. Thus, disaccharides 1-6 (FIG. 2) were selected as targets. These disaccharides contain either a D- or L-MTX residue (1-3 and 4-6, respectively) in an a-linkage to one of the three secondary hydroxyl groups of methyl α-D-mannopyranoside. These six disaccharides were synthesized with the goal of comparing their NMR data with that reported for this residue in the native polysaccharide to establish not only the absolute configuration of the modified pentose, but also its linkage position to the polysaccharide.

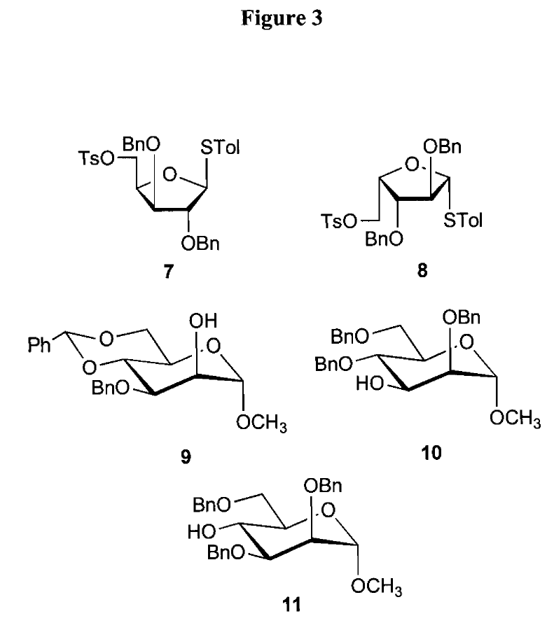

Synthesis: To synthesize these targets, a strategy was developed in which the methylthio group would be introduced near the end of the synthesis. This approach required the preparation of a series of six protected disaccharides with a leaving group at the primary position of the xylofuranose residue. It was envisioned that the five building blocks shown in FIG. 3 (7-11) could be used to assemble disaccharides 1-6. Mannopyranosides 9-11 were prepared as previously described (Nashed and Anderson (1976) Tetrahedron Lett. 3503-3506; and Koto (1984) Bull. Chem. Soc. Jpn. 57:3603-3604). The tosylated thioglycosides 7 and 8 were synthesized as described below.

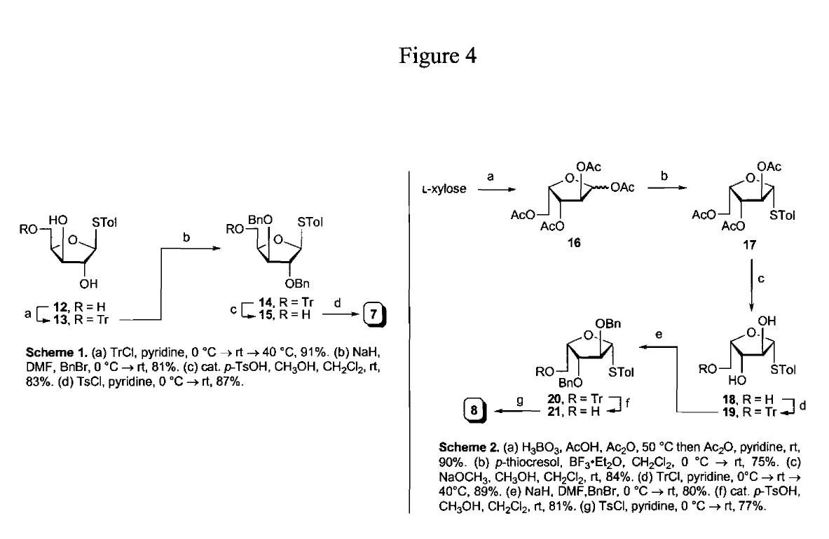

The preparation of 7 (Scheme 1; FIG. 4) began from thioglycoside triol 12 (Tilekar and Lowary (2004) Carbohydr. Res. 339:2895-2899), which was tritylated and benzylated under conventional conditions providing 14 in 74% yield over the two steps. The trityl group was then cleaved p-TsOH/CH₃OH) affording an 83% yield of alcohol 15. Subsequent tosylation of 15 yielded 7 in 87% yield.

The synthesis of the enantiomeric thioglycoside, 8, is illustrated in Scheme 2 (FIG. 4). In the first step, L-xylose (Ness (1962) Methods Carbohydr. Chem. 1:90-93) was converted to the corresponding furanose tetraacetate 16 in excellent yield (90%) using the boric acid-mediated approach developed by Furneaux ((2000) J. Chem. Soc. Perkin Trans. 1:2011-2014). Peracetate 16, obtained as an ˜2:1 anomeric mixture, was converted to thioglycoside 17 in 75% yield upon reaction with p-thiocresol and boron trifluoride etherate. Deacetylation of 17 with sodium methoxide in methanol provided, in 84% yield, triol 18, the enantiomer of 12. The synthesis of 8 from 18 was done via a sequence identical to that used for the preparation of 7 from 12. Thus, tritylation of 18 yielded 19 (89% yield), which was then benzylated affording 20 in 80% yield. Cleavage of the trityl group in 20 provided alcohol 21, which was then tosylated affording thioglycoside 8 in 62% yield over the two steps.

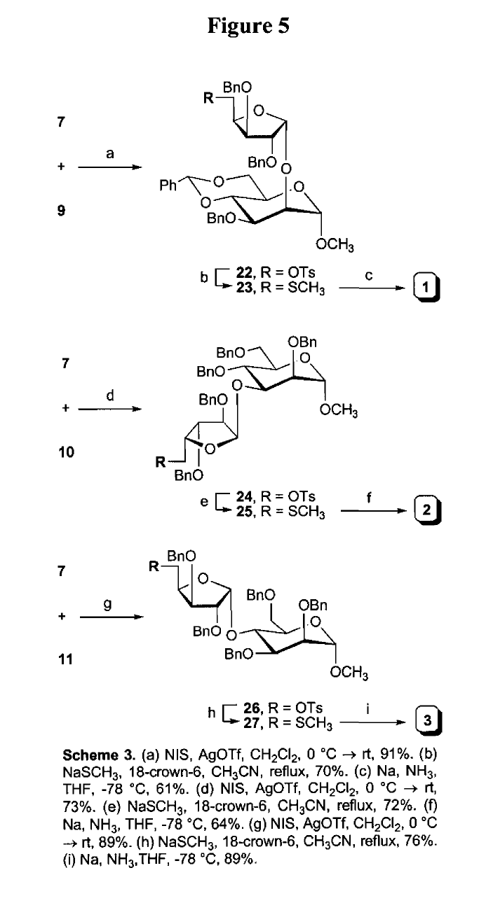

With sufficient quantities of building blocks 7-11 in hand, their coupling to provide disaccharides proceeded without significant problems. Shown in Scheme 3 (FIG. 5) is the synthesis of disaccharides containing the D-enantiomer of MTX (1-3).

The first step towards disaccharide 1 involved the reaction of thioglycoside 7 with mannopyranoside 9, in the presence of N-iodosuccinimide and silver triflate. The product produced from this reaction, disaccharide 22, was produced in 91% yield as an inseparable 87:13 α:β mixture of glycosides. The stereochemistry of the nascent glycosidic linkage could be readily established by NMR-spectroscopy. In the major product, the coupling constant between H-1 and H-2 (³J_{1,2}) in the xylofuranose residue was 4.3 Hz as would be expected for a 1,2-cis furanoside (Cyr and Perlin (1979) Can. J. Chem. 57:250425 11). In contrast, in the minor isomer, H-1 of the xylofuranose residue appeared as a singlet, consistent with the 1,2-trans furanoside stereochemistry. Further support for the anomeric stereochemistry of the xylofuranose residue was obtained from the ¹³C-NMR spectrum of the product. For the major isomer, the anomeric carbon resonance appeared at 101.4 ppm, whereas in the minor isomer this resonance appeared at 106.1. Again, both of these data support the a-stereochemistry of the major product. These same two NMR parameters were used to establish the stereochemistry of the xylofuranosyl bond in all the disaccharides synthesized.

All glycosylations reported here were highly ac-selective providing, at worst, an 87:13 α:β ratio of glycosides. Indeed, in some reactions, none of the β-glycoside product was isolated. This high selectivity for the 1,2-cis furanoside is in contrast to the synthesis of other 1,2-cis furanosides (e.g., β-arabinofuranosides), which often is plagued with modest anomeric selectivity (Yin and Lowary (2001) Tetrahedron Lett. 42:5829-5832) except under highly optimized conditions (Yin et al. (2002) J. Org. Chem. 67:892-903; and Lee et al. (2005) Org. Lett. 7:3263-326). The origin of the high selectivities observed in glycosylations with 7 and 8 as compared to other furanoside glycosylating agents containing non-participating groups on O-2 was unclear. It is plausible that the α-xylofuranoside product is favored by the kinetic anomeric effect (Juaristi and Cuevas, The Anomeric Effect CRC Press: Boca Raton, Fla., 1995, pp. 182-194), although in the absence of a detailed conformational study of the putative oxocarbenium ion involved in these reactions this is only a hypothesis.

Because the separation of 22 from the corresponding β-isomer was not possible, the mixture was submitted to the next reaction, in which the methylthio group was introduced. This reaction was done by heating 22 together with sodium thiomethoxide and 1 8-Crown-6 in acetonitrile at reflux. The expected product, 23, was produced in 70% yield, again contaminated with traces of its β-glycoside isomer. That the introduction of the methylthio group had occurred was obvious from the NMR spectra of 23. In the ¹H NMR spectrum, the signals for the protons on C-5 of the xylofuranose residue were significantly upfield (2.85 and 2.70 ppm) of their position in the ¹H NMR spectrum of 22 (4.10 and 4.29 ppm). In addition, in the ¹³C NMR spectrum of 23, the resonance for the xylofuranose C-5 appeared at 34.10 ppm, consistent with its linkage to sulfur. Finally, as expected, a methyl group bound to sulfur was apparent in both the ¹H and ¹³C spectra (resonances as 2.16 and 16.5 ppm, respectively). Similar features were observed in the NMR spectra for all products of these substitution reactions.

With the methylthio group in place, the final step in the synthesis of 1 was the cleavage of the benzyl ethers and the benzylidene acetal, which was done by dissolving metal reduction. Thus, treatment of a solution of 23 in THF at −78° C. with sodium and ammonia cleaved all protecting groups. Following purification, disaccharide 1 was isolated in 61% yield.

The synthesis of 2 followed a similar sequence to that used for the preparation of 1. Glycosylation of 10 with 7 promoted by N-iodosuccinimide and silver triflate gave disaccharide 24, as an inseparable mixture with the 0-glycoside and small amounts of hydrolyzed 7. The mixture was then subjected to the thiolate substitution reaction, which gave, following chromatography, 25 as a pure compound in 53% overall yield from 10. Removal of the benzyl ethers upon treatment of 25 with sodium and liquid ammonia in THF proceeded uneventfully, yielding 2 in 64% yield.

The same series of transformations was used to convert 11 and 7 into disaccharide 3. The coupling of 1 1 and 7 under standard conditions gave the expected disaccharide 26, which, following chromatography, was also contaminated with traces of hydrolyzed 7. This partially pure product was then reacted with sodium thiomethoxide to give 27 in 66% yield from 11. Disaccharide 3 was obtained in 89% yield upon treatment of 27 with sodium in liquid ammonia.

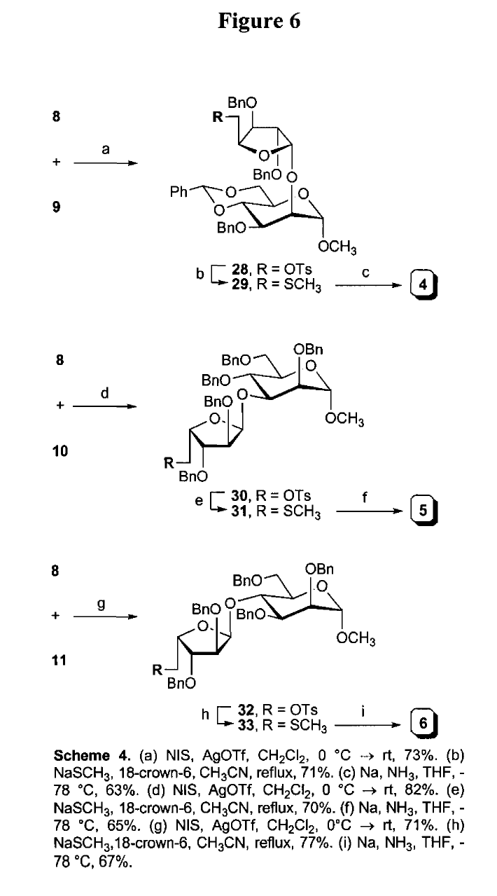

The synthesis of disaccharides containing an L-MTX residue (46) is shown in Scheme 4 (FIG. 6). The oligosaccharides were synthesized via the same routes used for the preparation of 1-3, by replacing donor 7 with 8. The protected disaccharides were thus obtained in yields of 71-82% upon reaction of 8 with one of acceptors 9-11. The resulting products 28, 30 and 32 were then converted to the methylthio analogs 29, 31 and 33 in 70-77% yield and subsequently deprotected by dissolving metal reduction, yielding 416 in 63-67% yield.

Synthesis Particulars

General Methods: Reactions were carried out in oven-dried glassware. Reaction solvents were distilled from appropriate drying agents before use. Unless stated otherwise, all reactions were carried out with stirring at room temperature under a positive pressure of argon and were monitored by TLC on silica gel 60 F₂₅₄ (0.25 mm, E. Merck). Spots were detected under UV light or by charring with acidified p-anisaldehyde solution in ethanol. In the processing of reaction mixtures, solutions of organic solvents were washed with equal amounts of aqueous solutions. Organic solutions were concentrated under vacuum at <40° C. All column chromatography was performed on silica gel (4060 μM) or Jatrobeads, which refers to a beaded silica gel 6RS-8060, manufactured by latron Laboratories (Tokyo). In all cases the ratio between adsorbent and crude product ranged from 100 to 50:1 (w/w). Optical rotations were measured at 22±2° C. and in units of degrees.mL/g-dm. ¹H NMR spectra were recorded at 400 or 500 MHz, and chemical shifts were referenced to either tetramethylsilane (0.0, CDCl₃), CD₃O H (4.78, CD₃OD), or 3-(trimethylsilyl)-propionic acid, sodium salt (0.0, D₂O). ¹³C NMR spectra were recorded at 100 or 125 MHz, and ¹³C chemical shifts were referenced to internal CDCl₃ (77.23, CDCl₃), CD₃OD (48.9, CD₃OD), or 3-(trimethylsilyl)-propionic acid, sodium salt (0.0, D₂O). ¹H data are reported as though they were first order. Electrospray mass spectra were recorded on samples suspended in mixtures of THF with CHOH and added NaCl.

Methyl 2-O-(5-deoxy-5-methylthio-α-D-xylofuranosyl)-α-D-mannopyranoside (1): Disaccharide 23 (21 mg, 0.03 mmol) was dissolved in THF (5 mL) and the solution was cooled to −78° C. and then NH₃ (20 mL) was condensed into the flask using a dry ice trap. Sodium metal (80 mg) was added in three portions until a deep blue color persisted. The solution was stirred for 1.5 hours at −78° C. and then CHOH (2 mL) was added. The flask was warmed to room temperature and left open to the atmosphere overnight to allow the NH₃ to evaporate. The remaining solution was concentrated and the resulting residue was dissolved in a minimum amount of CHOH before being neutralized with glacial HOAc. The solution was again concentrated and the semisolid residue was purified by column chromatography on latrobeads (85:15, CH₂Cl₂:CH₃OH) to afford 1 (6 mg, 61%) as a foam (data for major isomer). R_{f} 0.24 (85:15, CH₂Cl₂:CH₃OH); [α]_{D}+75.2 (c 0.4, CH₃OH); ¹H NMR (500 MHz, D₂O, δ_{H}) 5.30 (d, 1 H, J=4.5 Hz, H-1′), 4.93 (d, 1 H, J=1.7 Hz, H-1), 4.40 (ddd, 1 H, J=4.8, 5.0, 8.6 Hz, H-4′), 4.27 (dd, 1 H, J=4.2, 4.5 Hz, H-3′), 4.21 (dd, 1 H, J=4.5, 4.5 Hz, H-2′), 3.99 (dd, 1 H, J=1.7, 3.4 Hz, H-2), 3.89 (dd, 1 H, J=1.9, 12.3 Hz, H-6), 3.85 (dd, 1 H, J=3.4, 9.7 Hz, H-3), 3.80 (dd, 1 H, J=5.6, 12.3 Hz, H-6), 3.71 (dd, 1 H, J=9.7, 9.7 Hz, H-4), 3.63-3.60 (m, 1 H, H-5), 3.42 (s, 3 H, OCH₃), 2.80 (dd, 1 H, J=5.0, 13.8 Hz, H-5′), 2.69 (dd, 1 H, J=8.6, 13.8 Hz, H-5′), 2.18 (s, 3 H, SCH₃); ¹³C NMR (125 MHz, D₂O, δ_{C}) 105.8 (C-1′), 103.0 (C-1), 80.8 (C-2), 80.6 (C-4′), 80.4 (C-2′), 78.5 (C-3′), 75.3 (C-5), 73.2 (C-3), 69.6 (C-4), 63.5 (C-6), 57.8 (OCH₃), 35.6 (C-5′), 17.9 (SCH₃). HRMS (ESI) cared for (M+Na) C₁₃H₂₄O₉S: 379.1033, found 379.1032.

Methyl 3-O-(5-deoxy-5-methylthio-α-D-xylofuranosyl)-α-D-mannopyranoside (2): Prepared from 25 (24 mg, 0.03 mmol), liquid NH₃ (20 mL) and sodium metal (80 mg) in THF (5 mL) as described for 1, to afford 2 (7 mg, 64%) as a foam. R_{f} 0.4 (85:15, CH₂Cl₂:CH₃OH); [α]_{D}+106.6 (c 0.5, CH₃OH); ¹H NMR (500 MHz, D₂O, δ_{H}) 5.36 (d, 1 H, J=4.5 Hz, H-1′), 4.76 (s, 1 H, H-1), 4.43 (ddd, 1 H, J=5.3, 5.0, 8.4 Hz, H-4′), 4.29 (dd, 1 H, J=4.0, 5.3 Hz, H-3′), 4.20 (dd, 1 H, J=4.5, 4.0 Hz, H-2′), 4.14-4.11 (m, 1 H, H-2), 3.92-3.86 (m, 2 H, H-3, H-6), 3.82-3.75 (m, 2 H, H-4, H-6), 3.69-3.65 (m, 1 H, H-5), 3.42 (s, 3 H, OCH₃), 2.81 (dd, 1 H, J=5.0, 13.8 Hz, H-5′), 2.69 (dd, 1 H, J=8.4, 13.8 Hz, H-5′), 2.17 (s, 3 H, SCH₃); ¹³C NMR (125 MHz, D₂O, δ_{C}) 105.4 (C-1′), 103.5 (C-1), 81.6 (C-2), 80.6 (C-4′), 80.4 (C-2′), 78.5 (C-3′), 7.54 (C-5), 72.9 (C-3), 68.6 (C-4), 63.7 (C-6), 57.7 (OCH₃), 35.6 (C-5′), 17.8 (SCH₃). HRMS (ESI) calcd for (M+Na) C₁₃H₂₄O₉S: 379.1033, found 379.1032.

Methyl 4-O-(5-deoxy-5-methylthio-α-D-xylofuranosyl)-α-D-mannopyranoside (3): Prepared from 27 (0.39 g, 0.48 mmol), liquid NH3 (35 mL) and sodium metal (75 mg, 3.26 mmol) in THF (5 mL) as described for 1, to afford 3 (0.15 g, 89%) as a foam; R_{f} 0.48 (85:15, CH₂Cl₂:CH₃OH); [α]_{D}+109.5 (c 0.33, CH₃OH); ¹H NMR (500 MHz, D₂O, δ_{H}) 5.41 (d, 1 H, J=4.4 Hz, H-1′), 4.76 (s, 1 H, H-1), 4.38 (ddd, 1 H, J=5.0,4.8, 8.4 Hz, H-4′), 4.26 (dd, 1 H, J=4.2, 5.0 Hz, H-3′), 4.21 (dd, 1 H, J=4.4, 4.2 Hz, H-2′), 3.94-3.88 (m, 3 H, H-2, H-4, H-6), 3.83-3.75 (m, 2 H, H-3, H-6), 3.72-3.66 (m, 1 H, H-5), 3.41 (s, 3 H, OCH₃), 2.80 (dd, 1 H, J=4.8, 13.8 Hz, H-5), 2.68 (dd, 1 H, J=8.4, 13.8 Hz, H-5′), 2.18 (s, 3 H, SCH₃); ¹³C NMR (125 MHz, D₂O, δ_{C}) 105.3 (C-1′), 103.7 (C-1), 80.6 (C-4′), 79.4 (C-2′), 78.4 (C-3′), 76.9 (C-2), 74.0 (C-5), 73.5 (C-3), 73.0 (C-4), 63.9 (C-6), 57.6 (OCH₃), 35.8 (C-5), 17.8 (SCH₃). HRMS (ESI) calcd for (M+Na) C₁₃H₂₄O₉S: 379.1033, found 379.1032.

Methyl 2-O-(5-deoxy-5-methylthio-α-L-xylofuranosyl)-α-D-mannopyranoside (4): Prepared from 29 (25 mg, 0.03 mmol), liquid NH3 (20 mL) and sodium metal (80 mg) in THF (5 mL) as described for 1, to afford 4 (8 mg, 63%) as a foam. R_{f} 0.39 (85:15, CH₂Cl₂:CH₃OH); [α]_{D}−13.4 (c 0.1, CHOH); ¹H NMR (500 MHz, D₂O, δ_{H}) 5.25 (d, 1 H, J=4.4 Hz, H-1′), 4.88 (s, 1 H, H-1), 4.47 (ddd, 1 H, J=5.0, 4.9, 8.4 Hz, H-4′), 4.30 (dd, 1 H, J=4.9, 4.2 Hz, H-3′), 4.19 (dd, 1 H, J=4.2, 4.4 Hz, H-2′), 4.05-4.02 (m, 1 H, H-2), 3.88 (dd, 1 H, J=1.9, 12.0 Hz, H-6), 3.83 (dd, 1 H, J=3.5, 9.8 Hz, H-3), 3.80 (dd, 1 H, J=5.0, 12.0 Hz, H-6), 3.70 (dd, 1 H, J=9.8, 9.8 Hz, H-4), 3.65-3.60 (m, 1 H, H-5), 3.41 (s, 3 H, OCH₃), 2.79 (dd, 1 H, J=5.0, 13.8 Hz, H-5′), 2.68 (dd, 1 H, J=8.4, 13.8 Hz, H-5′), 2.16 (s, 3 H, SCH₃); ¹³C NMR (125 MHz, D₂O, δ_{C}) 103.0 (C-1′), 101.4 (C-1), 80.4 (C-4′), 80.0 (C-2′), 79.2 (C-2), 78.3 (C-3′), 75.4 (C-5), 72.8 (C-3), 69.7 (C-4), 63.3 (C-6), 57.7 (OCH₃), 35.6 (C-5′), 17.7 (SCH₃). HRMS (ESI) calcd for (M+Na) C₁₃H₂₄O₉S: 379.1033, found 379.1031.

Methyl 3-O-(5-deoxy-5-methylthio-α-L-xylofuranosyl)-α-D-mannopyranoside (5): Prepared from 31 (32 mg, 0.04 mmol), liquid NH₃ (25 mL) and sodium metal (80 mg) in THE (5 mL) as described for 1, to afford 5 (9 mg, 65%) as a foam. R_{f} 0.44 (85:15, CH₂Cl₂CH₃OH); [α]_{D}−18.4 (c 0.28, CH₃OH); ¹H NMR (500 MHz, D₂O, δ_{H}) 5.27 (d, 1 H, J=4.4 Hz, H-1′), 4.80 (d, 1 H, J=1.8 Hz, H-1), 4.47 (ddd, 1 H, J=5.2, 5.6, 8.3 Hz, H-4′), 4.31 (dd, 1 H, J=4.6, 5.6 Hz, H-3′), 4.20 (dd, 1 H, J=4.4, 4.6 Hz, H-2′), 4.12-4.10 (dd, 1 H, J=1.8, 3.2 Hz, H-2), 3.94-3.87 (m, 2 H, H-3, H-6), 3.81-3.73 (m, 2 H, H-4, H-6), 3.70-3.64 (m, 1 H, H-5), 3.42 (s, 3 H, OCH₃), 2.80 (dd, 1 H, J=5.2, 13.8 Hz, H-5′), 2.68 (dd, 1 H, J=8.3, 13.8 Hz, H-5′), 2.16 (s, 3 H, SCH₃); ¹³C NMR (125 MHz, D₂O, δ_{C}) 103.4 (C-1′), 101.3 (C-1), 80.2(4) (C-4′), 80.2(1) (C-2′), 79.6 (C-2), 78.4 (C-3), 75.3 (C-5), 67.9(9) (C-3), 67.9(8) (C-4), 63.8 (C-6), 57.6 (OCH₃), 35.7 (C-5′), 17.7 (SCH₃). HRMS (ESI) calcd for (M+Na) C₁₃H₂₄O₉S: 379.1033, found 379.1031.

Methyl 4-O-(5-deoxy-5-methylthio-α-L-xylofuranosyl)-α-D-mannopyranoside (6): Prepared from 33 (32 mg, 0.04 mmol), liquid NH₃ (30 mL) and sodium metal (90 mg) in THF (5 mL) as described for 1, to afford 6 (9 mg, 67%) as a foam. R_{f} 0.5 (85:15, CH₂Cl₂CH₃OH); [α]_{D}+1.3 (c 0.5, CHOH); ¹H NMR (500 MHz, D₂O, δ_{H}) 5.21 (d, 1 H, J=4.4 Hz, H-1′), 4.77 (d, 1 H, J=1.8 Hz, H-1), 4.47 (ddd, 1 H, J=5.2, 4.9, 8.6 Hz, H-4′), 4.28 (dd, 1 H, J=5.2, 4.6 Hz, H-3′), 4.20 (dd, 1 H, J=4.6, 4.4 Hz, H-2′), 3.99 (dd, 1 H, J=5.8, 3.4 Hz, H-2), 3.90-3.86 (m, 2 H, H-3, H-6), 3.85 3.76 (m, 2 H, H-4, H-6), 3.75-3.71 (m, 1 H, H-5), 3.41 (s, 3 H, OCH₃), 2.80 (dd, 1 H, J=4.9, 13.8 Hz, H-5′), 2.68 (dd, 1 H, J=8.6, 13.8 Hz, H-5′), 2.16 (s, 3 H, SCH₃); ¹³C NMR (125 MHz, D₂O, δ_{C}) 104.6 (C-1′), 103.6 (C-1), 80.3 (C-4′), 79.6 (C-2′), 78.6 (C-2), 78.2 (C-3′), 74.1 (C-5), 72.7 (C-3), 72.1 (C-4), 63.3 (C-6), 57.7 (OCH₃), 35.8 (C-5′), 17.8 (SCH₃). HRMS (ESI) calcd for (M+Na) C₁₃H₂₄O₉S: 379.1033, found 379.1034.

p-Tolyl 2,3-di-O-benzyl-5-O-toluenesulfonyl-1-thio-β-D-xylofuranoside (7): To a solution of 15 (1.1 g, 2.52 mmol) in pyridine (6 mL) at 0° C. was added toluenesulfonyl chloride (0.625 g, 3.28 mmol). The reaction mixture was stirred at room temperature for 12 hours and then poured into ice water (40 mL) and extracted with CH₂Cl₂ (2×40 mL). The combined CH₂Cl₂ extracts were washed with 7% aq. CuSO₄ solution (3×75 mL), water (1×75 mL), dr (Na₂SO₄) and concentrated to a syrup that was purified by column chromatography (12:1, hexanes:EtOAc) to afford 7 (1.29 g, 87%) as a syrup. R_{f} 0.38 (4:1, hexanes:EtOAc); [α]_{D}−70.9 (c 1.0, CHCl₃); ¹H NMR (500 MHz, CDCl₃, δ_{H}) 7.80-7.75 (m, 2 H), 7.40-7.20 (m, 14 H), 7.10-7.05 (m, 2 H), 5.25 (d, 1 H, J=2.8 Hz), 4.56 (d, 1 H, J=11.8 Hz), 4.48 (dd, 2 H, J=8.8, 11.8 Hz), 4.41-4.34 (m, 3 H), 4.32-4.25 (m, 1 H), 4.07-4.02 (m, 2 H), 2.40 (s, 3 H), 2.32 (s, 3 H); ¹³C NMR (125 MHz, CDCl₃, δ_{C}) 144.7, 137.5, 137.2, 137.1, 132.8, 131.9 (2 C), 130.9, 129.8 (2 C), 129.7 (2 C), 128.5 (2 C), 128.4(7) (2 C), 128.1, 128.0 (2 C), 127.8 (5 C), 90.8, 86.2, 81.4, 79.2 72.1(2 C), 68.2, 21.6, 21.1. HRMS (ESI) calcd for (M+Na) C₃₃H₃₄O₆S₂: 613.1689, found 613.1690.

p-Tolyl 2,3-di-O-benzyl-5-O-toluenesulfonyl-1-thio-β-L-xylofuranoside (8): Prepared from 21 (0.9 g, 2.06 mmol) and toluenesulfonyl chloride (0.51 g, 2.68 mmol) in pyridine (6 mL) as described for 7, to afford 8 (0.936 g, 77%) as a syrup. R_{f} 0.38 (4:1, hexanes:EtOAc); [α]_{D}+67.8 (c 1.0, CHCl₃); ¹H NMR (500 MHz, CDCl₃, δ_{H}) 7.80-7.75 (m, 2 H), 7.40-7.20 (m, 14 H), 7.10-7.05 (m, 2 H), 5.25 (d, l H, J=2.8 Hz), 4.56 (d, 1 H, J=11.8 Hz), 4.48 (dd, 2 H, J=8.8, 11.8 Hz), 4.41-4.34 (m, 3 H), 4.32-4.25 (m, 1 H), 4.07-4.02 (m, 2 H), 2.40 (s, 3 H), 2.32 (s, 3 H); ¹³C NMR (125 MHz, CDCl₃, δ_{C}) 144.7, 137.5, 137.2, 137.1, 132.8, 131.9 (2 C), 130.9, 129.8 (2 C), 129.7 (2 C), 128.5(2) (2 C), 128.4(7) (2 C), 128.1, 128.0, 127.9(6), 127.8 (5 C), 90.8, 86.2, 81.4, 79.2, 72.1(2 C), 68.2, 21.6, 21.1. HRMS (ESI) calcd for (M+Na) C₃₃H₃₄O₆S₂: 613.1689, found 613.1691.

p-Tolyl 5-O-trityl-1-thio-β-D-xylofuranoside (13): To a solution of 12 (30) (1.2 g, 4.67 mmol) in pyridine (8 mL) at room temperature was added DMAP (0.183 g, 1.5 mmol) followed by trityl chloride (1.63 g, 5.84 mmol). The reaction mixture was stirred at 45° C. for 14 hours and then poured into ice water (30 mL) and extracted with CH₂Cl₂ (2×30 mL). The combined CH₂Cl₂ extracts were washed with 7% aq. CuSO₄ solution (3×75 mL), water (1×75 mL, dried (Na₂SO₄) and concentrated to a syrup that was purified by column chromatography (4:1, hexanes:EtOAc) to afford 13 (2.12 g, 91%) as a syrup. R_{f} 0.5 (1;1, hexanes:EtOAc); [α]_{D}−81.6 (c 1.0, CHCl₃); ¹H NMR (500 MHz, CDCl₃, δ_{H}) 7.53-7.40 (m, 8 H), 7.35-7.20 (m, 9 H), 7.10-7.14 (m, 2 H), 5.23 (d, 1 H, J=3.7 Hz), 4.34-4.28 (m, 2 H), 4.19 (dd, 1 H, J=3.0, 5.1 Hz), 3.51 (dd, 1 H, J=4.6, 10.4 Hz), 3.32-3.27 (m, 2 H), 2.33 (s, 3 H); ¹³C NMR (125 MHz, CDCl₃, δ_{C}) 143.4 (3 C), 137.7, 132.3 (3 C), 130.6, 129.8 (3 C), 128.6 (4 C), 128.0 (4 C), 127.2 (3 C), 91.4, 87.6, 82.0, 80.2, 78.1, 62.9, 21.1. HRMS (ESI) calcd for (M+Na) C₃₁H₃₀O₄S: 521.1757, found 521.1758.

p-Tolyl 2,3-di-O-benzyl-5-O-trityl-1-thio-β-D-xylofuranoside (14): To a solution of 13 (2.0 g, 4.0 mmol) in DMF (8 mL) at 0° C., was added NaH (60% suspension in oil, 0.42 g, 10.42 mmol) in portions. The mixture was stirred for 5 minutes before benzyl bromide (1.25 mL, 10.5 mmol) was added dropwise. After stirring for 4 h, the reaction mixture was poured into ice water (80 mL) and extracted with CH₂Cl₂ (2×40 mL). The combined CH₂Cl₂ extracts were washed with water (2×40 mL), dried (Na₂SO₄) and concentrated to a syrup that was purified by column chromatography (12:1, hexanes:EtOAc) to afford 14 (2.2 g, 81%) as a syrup. R_{f} 0.46 (5.6:1, hexanes:EtOAc); [α]_{D}−45.6 (c 1.0, CHCl₃); ¹H NMR (500 MHz, CDCl₃, δ_{H}) 7.50-7.05 (m, 29 H), 5.34 (d, 1 H, J=2.8 Hz), 4.60 (d, 1 H, J=11.9 Hz), 4.52 (d, 1 H, J=12.2 Hz), 4.49 (d, 1 H, J=12.2 Hz), 4.40 (dd, 1 H, J=5.6, 10.6 Hz), 4.32 (d, 1 H, J=12.2 Hz), 4.10 (dd, 1 H, J=1.7, 1.7 Hz), 4.00 (dd, 1 H, J=1.7, 4.5 Hz), 3.60 (dd, 1 H, J=6.4, 9.6 Hz), 3.32 (dd, 1 H, J=5.5, 9.6 Hz), 2.31 (s, 3 H); ¹³C NMR (125 MHz, CDCl₃, δ_{C}) 144.1 (3 C), 137.7, 137.4, 137.1, 131.7, 131.6 (3 C), 129.6 (2 C), 128.8 (4 C), 128.5 (2 C), 128.3 (2 C), 128.2, 127.9, 127.8(4), 127.8(2), 127.7(4) (4 C), 127.7(2), 127.7, 127.6 (2 C), 127.3, 126.9 (3 C), 90.5, 86.8, 86.8, 81.6, 81.4, 72.0, 71.7, 62.5, 21.1. HRMS (ESI) calcd for (M+Na) C₄₅H₄₂O₄S: 701.2696, found 701.2698.

p-Tolyl 2,3-di-O-benzyl-1-thio-β-D-xylofuranoside (15): To a solution of 14 (2.1 g, 3.09 mmol) in CH₂Cl₂:CHOH (7:3, 30 mL) at room temperature was added p-TsOH (40 mg). The mixture was stirred for 15 h, neutralized with Et₃N and concentrated to a syrup that was purified by column chromatography (4:1, hexanes:EtOAc) to afford 15 (1.12 g, 83%) as a syrup. R_{f}0.21 (4:1, hexanes:EtOAc); [α]_{D}−82.7 (c 1.0, CHCl₃); ¹H NMR (500 MHz, CDCl₃, δ_{H}) 7.45-7.25 (m, 12 H), 7.15-7.10 (m, 2 H), 5.32 (d, 1 H, J=4.0 Hz), 4.72 (d, 1 H, J=11.8 Hz), 4.60 (d, 1 H, J=11.8 Hz), 4.58 (d, 1 H, J=11.8 Hz), 4.45 (d, 1 H, J=11.8 Hz), 4.27 (dd, 1 H, J=5.2, 10.5 Hz), 4.21-4.16 (m, 2 H), 3.92-3.82 (m, 2 H), 2.33 (s, 3 H); ¹³C NMR (125 MHz, CDCl₃, δ_{C}) 137.7, 137.4, 137.3, 132.2 (2 C), 130.6, 129.8 (2 C), 128.6 (2 C), 128.5 (2 C), 128.0(2), 128.0(2), 128.0(1), 127.9 (2 C), 127.7 (2 C), 90.1, 86.5, 83.0, 81.1, 72.4, 72.2, 61.7, 21.1. HRMS (ESI) calcd for (M+Na) C₂₆H₂₈O₄S: 459.1600, found 459.1600.

1,2,3,5-Tetra-O-acetyl-L-xylofuranose (16): L-Xylose (4.17 g, 27.8 mmol), boric acid (3.8 g, 60.7 mmol) and acetic acid (95 mL) were stirred at 50° C. for 1 hour before acetic anhydride (95 mL) was added. The mixture was heated at 50° C. for 16 hours and then cooled to rt. The boric acid was removed as trimethyl borate by the addition of methanol (20 mL) and in vacuo concentration of the resulting mixture to 100 mL and then the addition of methanol (10 mL) and concentration in vacuo to 50 mL (repeated twice). Acetic anhydride (100 mL) and pyridine (100 mL) were added and the solution was stirred at room temperature for 2 hours. Ice (˜250 g) was added and the mixture was stirred for 1 hour and then extracted with CH₂Cl₂ (3×150 mL). The combined CH₂Cl₂ extracts were washed with 7% aq. CuSO₄ solution (3×300 mL), water (2×250 mL), dried (Na₂SO₄) and concentrated to a syrup that was purified by column chromatography (7:3, hexanes:EtOAc) to afford 16 (7.96 g, 90%, α:β, 1:1.8) as a syrup. R_{f}0.2 (7:3, hexanes:EtOAc); ¹H NMR (500 MHz, CDCl₃, δ_{H}) 6.42 (d, 0.35 H, J=4.6 Hz), 6.10 (s, 0.65 H), 5.52 (dd, 0.35 H, J=6.5, 6.5 Hz), 5.36 (dd, 0.65 H, J=1.7, 5.6 Hz), 5.30 (dd, 0.35 H, J=4.6, 6.2 Hz), 5.20 (d, 0.65 H, J=1.0 Hz), 4.67-4.60 (m, 1 H), 4.27-4.18 (m, 1.65 H), 4.12 (dd, 0.35 H, J=4.2, 12.2 Hz), 2.12 (s, 2H), 2.11 (s, 2 H), 2.09 (s, 3 H), 2.07 (s, 3 H), 2.06 (s, 2 H); ¹³C NMR (125 MHz, CDCl₃, δ_{C}) 170.5, 170.3, 169.6, 169.5, 169.3, 169.2, 169.1, 98.8, 92.8, 79.9, 79.4(1), 75.3(9), 75.3, 74.3, 73.8, 62.3, 61.6, 21.0, 20.9, 20.8, 20.7, 20.6, 20.5, 20.4. HRMS (ESI) calcd for (M+Na) C₁₃H₁₈O₉: 341.0843, found 341.0845.

p-Tolyl 2,3,5-tri-O-acetyl-1-thio-β-L-xylofuranoside (17): To a solution of 16 (3.0 g, 9.43 mmol) in CH₂Cl₂ (60 mL) at −20° C. was added p-thiocresol (1.29 g, 10.38 mmol) followed by BF_{3.Et}₂O (2.96 mL, 23.58 mmol) dropwise over 6 minutes. The reaction mixture was stirred at −20° C. for 6 h, neutralized (at −20° C.) with Et₃N and concentrated to a syrup that was purified by column chromatography (4:1, hexanes:EtOAc,) to afford 17 (2.3 g, 75%, α:β, 1:49) as a syrup. R_{f}0.37 (7:3, hexanes:EtOAc); data for major isomer; [α]_{D}+83.8 (c 0.5, CHCl₃); ¹H NMR (400 MHz, CDCl₃, δ_{H}) 7.44 (d, 2 H, J=8.1 Hz), 7.14 (d, 2 H, J=8.1 Hz), 5.30 (dd, 1 H, J=2.2, 5.1 Hz), 5.26 (dd, 1 H, J=2.2, 3.3 Hz), 5.18 (d, 1 H, J=3.3 Hz), 4.45 (ddd, 1 H, J=5.1, 5.1, 6.5 Hz), 4.32 (dd, 1 H, J=5.1, 11.7 Hz), 4.24 (dd, 1 H, J=6.5, 11.7 Hz), 2.33 (s, 3 H), 2.09 (s, 3 H), 2.07 (s, 3 H), 2.05 (s, 3 H); ¹³C NMR (100 MHz, CDCl₃, δ_{C}) 170.5, 169.6, 169.2, 138.2, 133.3 (2 C), 129.7 (2 C), 129.3, 90.2, 80.4, 78.4, 75.2, 62.0, 21.1, 20.8, 20.7, 20.6. HRMS (ESI) calcd for (M+Na) C₁₈H₂₂O₇S: 405.0978, found 405.0977.

p-Tolyl 1-thio-β-L-xylofuranoside (18): To a solution of 17 (2.0 g, 5.24 mmol) in CH₂Cl₂:CHOH (7:3, 30 mL) was added NaOCH₃ (0.16 g, 3.0 mmol). The mixture was stirred at room temperature for 7 hours then neutralized with glacial HOAc and concentrated to a syrup that was purified by column chromatography (3:7, hexanes:EtOAc) to afford 18 (1.13 g, 84%) as a syrup; R_{f}0.22 (3:7, hexanes:EtOAc); [α]_{D}+151.2 (c 0.5, CH₃OH); ¹H NMR (500 MHz, CD₃OD, δ_{H}) 7.40 (d, 2 H, J=8.2 Hz), 7.12 (d, 2 H, J=8.2 Hz), 5.06 (d, 1 H, J=3.7 Hz), 4.16-4.10 (m, 2 H), 4.06 (dd, 1 H, J=2.5, 3.7 Hz), 3.82 (dd, 1 H, J=4.3, 11.5 Hz), 3.74 (dd, 1 H, J=5.9, 11.5 Hz), 2.29 (s, 3 H); ¹³C NMR (125 MHz, CD₃OD, δ_{C}) 138.4, 133.3, 132.7 (2 C), 130.6 (2 C), 93.5, 83.9, 83.5, 77.9, 62.2, 21.1. HRMS (ESI) calcd for (M+Na) C₁₂H₁₆O₄S: 279.0661, found 279.0659.

p-Tolyl 5-O-trityl-1-thio-β-L-xylofuranoside (19): Prepared from 18 (1.05 g, 4.09 mmol), DMAP (0.123 g, 1.0 mmol) and trityl chloride (1.425 g, 5.11 mmol) in pyridine (7 mL) as described for 13, to afford 19 (1.814 g, 89%) as a syrp. R_{f}0.5 (1:1, hexanes:EtOAc); [α]_{D}+88.6 (c 1.0, CHCl₃); ¹H NMR (500 MHz, CDCl₃, δ_{H}) 7.53-7.40 (m, 8 H), 7.35-7.20 (m, 9 H), 7.10-7.14 (m, 2 H), 5.23 (d, 1 H, J=3.7 Hz), 4.34-4.28 (m, 2 H), 4.19 (ddd, 1 H, J=3.0, 2.2, 5.2 Hz), 3.51 (dd, 1 H, J=4.6, 10.4 Hz), 3.32-3.27 (m, 2 H), 2.33 (s, 3 H); ¹³C NMR (125 MHz, CDCl₃, δ_{C}) 143.4 (3 C), 137.7, 132.3 (3 C), 130.6, 129.8 (3 C), 128.6 (4 C), 128.0 (4 C), 127.2 (3 C), 91.4, 87.6, 82.0, 80.2, 78.1, 62.9, 21.1. HRMS (ESI) calcd for (M+Na) C₃₁H₃₀O₄S: 521.1757, found 521.1753.

p-Tolyl 2,3-di-O-benzyl-5-O-trityl-1-thio-β-L-xylofuranoside (20): Prepared from 19 (1.8 g, 3.60 mmol), NaH (0.374 g, 9.36 mmol) and benzyl bromide (1.1 mL, 9.36 mmol) in DMF (9 mL) as described for 14, to afford 20 (1.96 g, 80%) as a syrup. R_{f}0.46 (5.6:1, hexanes:EtOAc,); [α]D+73.9 (c 1.2, CHCl₃); ¹H NMR (500 MHz, CDCl₃, δ_{H}) 7.50-7.05 (m, 29 H), 5.34 (d, 1 H, J=2.8 Hz), 4.60 (d, 1 H, J=11.9 Hz), 4.50 (d, 1 H, J=11.9 Hz), 4.48 (d, 1 H, J=12.2 Hz), 4.40 (dd, 1 H, J=5.7, 10.6 Hz), 4.32 (d, 1 H, J=12.2 Hz), 4.10 (dd, 1 H, J=1.7, 1.7 Hz), 4.0 (dd, 1 H, J=1.7, 4.5 Hz), 3.60 (dd, 1 H, J=6.4, 9.6 Hz), 3.32 (dd, 1 H, J=5.5, 9.6 Hz), 2.31 (s, 3 H, CH₃); ¹³C NMR (125 MHz, CDCl₃, δ_{C}) 144.1 (3 C), 137.7, 137.4, 137.1, 131.7, 131.6 (3 C), 129.6 (2 C), 128.8 (4 C), 128.5 (2 C), 128.2(9) (2 C), 128.2(5), 127.9, 127.8(4), 127.8(2), 127.7(4) (4 C), 127.7(2), 127.7, 127.6 (2 C), 127.3, 126.9 (3 C), 90.5, 86.8, 86.8, 81.6, 81.4, 72.0, 71.7, 62.5, 21.1. HRMS (ESI) calcd for (M+Na) C₄₅H₄₂O₄S: 701.2696, found 701.2695.

p-Tolyl 2,3-di-O-benzyl-]-thio-β-L-xylofuranoside (21): Prepared from 20 (1.9 g, 2.80 mmol), and p-TsOH (40 mg) in CH₂Cl₂:CH₃OH (7:3, 30 mL) as described for 15, to afford 21 (0.99 g, 81%) as a syrup. R_{f}0.21 (4:1, hexanes:EtOAc); [α]_{D}+89.7 (c 0.5, CHCl₃); ¹H NMR (500 MHz, CDCl₃, δ_{H}) 7.45-7.25 (m, 12 H), 7.15-7.10 (m, 2 H), 5.32 (d, 1 H, J=4.0 Hz), 4.72 (d, 1 H, J=11.8 Hz), 4.60 (d, 1 H, J=11.8 Hz), 4.58 (d, 1 H, J=11.8 Hz), 4.45 (d, 1 H, J=11.8 Hz), 4.27 (dd, 1 H, J=5.2, 10.5 Hz), 4.21-4.16 (m, 2 H), 3.92-3.82 (m, 2 H), 2.33 (s, 3 H); ¹³C NMR (125 MHz, CDCl₃, δ_{C}) 137.7, 137.4, 137.3, 132.2 (2 C), 130.6, 129.8 (2 C), 128.6 (2 C), 128.5 (2 C), 128.0(2), 128.0(1), 127.9 (2 C), 127.7 (2 C), 90.1, 86.5, 83.0, 81.1, 72.4, 72.2, 61.7, 21.1. HRMS (ESI) calcd for (M+Na) C₂₆H₂₈O₄S: 459.1600, found 459.1601.

Methyl 2-O-(2, 3-di-O-benzyl-5-O-toluenesulfonyl-α-D-xylofuranosyl)-3-O-benzyl-4,6-O-benzylidene-α-D-mannopyranoside (22): Thioglycoside 7 (0.21 g, 0.35 mmol) and alcohol 9 (29) (0.11 g, 0.3 mmol) were dried over P₂O₅ under vacuum for 6 hours and then dissolved in CH₂Cl₂ (4 mL) and the resulting solution was cooled to 0° C. Powdered 4 Å molecular sieves (75 mg) were added and the suspension was stirred for 20 minutes at 0° C. before N-iodosuccinimide (96 mg, 0.42 mmol) and silver triflate (16 mg, 0.06 mmol) were added. The reaction mixture was stirred for 15 min, neutralized with Et₃N, diluted with CH₂Cl₂ (10 mL) and filtered though Celite. The filtrate was washed successively with sat. aq. sodium thiosulfate (3×15 mL), water (1×15 mL), dried (Na₂SO₄) and concentrated to a syrup that was purified by column chromatography (4:1, hexanes:EtOAc) to afford 22 (0.22 g, 91%), as a syrup. The product was an inseparable mixture of isomers (60 :β, 87:13), which was used in the next step; data provided for major isomer. R_{f}0.49 (7:3, hexanes:EtOAc); ¹H NMR (500 MHz, CDCl₃, δ_{H}) 7.77 (d, 2 H, J=8.4 Hz), 7.50-7.20 (m, 22 H), 5.42 (d, 1 H, J=4.3 Hz), 5.27 (s, 1 H), 4.88 (d, 1 H, J=11.5 Hz), 4.82 (d, 1 H,J=11.3 Hz), 4.69 (d, 1 H,J=11.6 Hz), 4.64 (d, 1 H, J=1.6 Hz), 4.64 (d, 1 H, J=12.0 Hz), 4.48 (d, 1 H, J=11.9 Hz), 4.46-4.39 (m, 1 H), 4.39-4.33 (m, 2 H), 4.29 (dd, 1 H, J=3.6, 11.0 Hz), 4.20 (d, 1 H, J=5.4 Hz), 4.13-4.07 (m, 2 H), 4.07-4.02 (m, 1 H), 3.96 (dd, 1 H, J=3.1, 9.8 Hz), 3.93 (dd, 1 H, J=4.3, 5.5 Hz), 3.75 (d, 2 H, J=7.1 Hz), 3.37 (s, 3 H), 2.43 (s, 3 H); ¹³C NMR (125 MHz, CDCl₃, δ_{C}) 144.7, 138.5, 138.1, 137.8, 137.7, 133.0, 129.7(4), 129.7(0), 128.8, 128.5, 128.3(7) (2 C), 128.3(5) (2 C), 128.3 (2 C), 128.1(8) (2 C), 128.1(5), 128.0, 127.9, 127.8, 127.6(9), 127.6(6), 127.5(9), 127.5(7), 127.5, 126.1, 126.0 (2 C), 101.4, 99.1, 97.5, 84.5, 81.4, 78.4, 74.4(4), 74.4(0), 72.5, 72.1(7), 72.1(5), 71.8, 68.9, 68.8, 64.1, 54.9, 21.6. HRMS (ESI) calcd for (M+Na) C₄₇H₅₀0₁₂S: 861.2915, found 861.2912.

Methyl 2-O-(2,3-di-O-benzyl-5-deoxy-5-methylthio-α-D-xylofuranosyl)-3-O-benzyl-4,6-O-benzylidene-α-D-mannopyranoside (23): To a solution of 22 (70 mg, 0.08 mmol) in CH₃CN (2 mL) was added 18-crown-6 (20 mg) followed by sodium thiomethoxide (13 mg, 0.24 mmol). The reaction mixture was heated at reflux for 12 hours and then cooled to room temperature before being diluted with CH₃CN (6 mL) and filtered through Celite. The filtrate was concentrated to a syrup that was purified by column chromatography (5.6:1, hexanes:EtOAc) to afford 23 (42 mg, 70.0%) as a syrup. The product was an inseparable mixture of isomers (α:β, 87:13), which was used in the next step; data provided for major isomer. R_{f}0.39 (4:1, hexanes:EtOAc); ¹H NMR (400 MHz, CDCl₃, δ_{H}) 7.557.20 (m, 20 H), 5.46 (d, 1 H, J=4.4 Hz), 5.30 (s, 1 H), 4.90 (d, 1 H, J=9.3 Hz), 4.87 (d, 1 H, J=9.0 Hz), 4.75 (d, 1 H, J=1.6 Hz), 4.70 (dd, 2 H, J=7.5, 11.6 Hz), 4.54 (d, 1 H, J=11.9 Hz), 4.48-4.40 (m, 2 H), 4.27 (dd, 2 H, J=4.7, 6.5 Hz), 4.22-4.16 (m, 2 H), 4.03-3.94 (m, 2 H), 3.78-3.74 (m, 2 H), 3.38 (s, 3 H), 2.85 (dd, 1 H, J=5.1, 13.8 Hz), 2.70 (dd, 1 H, J=7.9, 13.8 Hz), 2.16 (s, 3 H); ¹³C NMR (125 MHz, CDCl₃, δ_{C}) 138.6, 138.2, 138.1, 137.7, 128.8, 128.3(3) (3 C), 128.3(2) (3 C), 128.3 (128.2 (2 C), 127.7 (4 C), 127.6(1), 127.6(0), 127.5 (2 C), 126.0, 101.7, 101.6, 101.2, 84.1, 82.1, 79.4, 77.3, 76.2, 75.7, 73.7, 72.0, 71.5, 68.7, 63.9, 54.8, 34.1, 16.5. HRMS (ESI) calcd for (M+Na) C₄₁H₄₆O₉S: 737.2754, found 737.2750.

Methyl 3-O-(2,3-di-O-benzyl-5-O-toluenesulfonyl-α-D-xylofuranosyl)-2,4,6-tri-O-benzyl-α-D-mannopyranoside (24): Prepared from thioglycoside 7 (0.12 g, 0.2 mmol), alcohol 10(29) (67 mg, 0.14 mmol), N-iodosuccinimide (55 mg, 0.24 mmol) and silver triflate (10 mg, 0.04 mmol) in CH₂Cl₂ (3 mL) as described for 22, to afford 24 (98 mg, 73%) as a syrup. The product 24 could not be completely purified from ˜12% of the β-glycoside and some hydrolyzed donor and hence was used as such for the next step; data provided for major isomer. R_{f}0.33 (4:1, hexanes:EtOAc); [α]_{D}+67.5 (c 0.5, CHCl₃); ¹H NMR (500 MHz, CDCl₃, δ_{H}) 7.20 (d, 2 H, J=8.3 Hz), 7.40-7.14 (m, 25 H), 7.14-7.06 (m, 2 H), 5.20 (d, 1 H, J=4.2 Hz), 4.86 (d, 1 H, J=11.2 Hz), 4.82 (d, 1 H, J=11.6 Hz), 4.76 (d, 1 H, J=1.7 Hz), 4.69 (d, 1 H, J=8.4 Hz), 4.66 (d, 1 H, J=12.0 Hz), 4.60 (d, 1 H, J=12.0 Hz), 4.54 (d, I H, J=3.5 Hz), 4.51 (d, 1 H, J=11.3 Hz), 4.42 (d, 1 H, J=11.7 Hz), 4.38 (d, 1 H, J=8.1 Hz), 4.29-4.24 (m, 2 H), 4.18 (dd, 1 H, J=3.6, 10.5 Hz), 4.03 (dd, 2 H, J=3.2, 9.4 Hz), 4.00-3.94 (m, 1 H), 3.88-3.84 (m, 2 H), 3.80-3.70 (m, 3 H), 3.38 (s, 3 H), 2.40 (s, 3 H); ¹³C NMR (125 MHz, CDCl₃, δ_{C}) 144.6, 138.6(9), 138.4, 137.6(0), 137.6, 133.0, 129.7, 128.6, 128.5, 128.4, 128.3(9) (2 C), 128.3(3) (2 C), 128.2(5), 128.2(4) (2 C), 128.2, 128.0, 127.9, 127.8, 127.7, 127.6(4) (2 C), 127.6(3) (2 C), 127.5(9) (2 C), 127.5(7) (2 C), 127.5(5), 127.3(9), 127.3(6), 127.2, 127.0, 101.9, 98.7, 82.8, 81.0, 80.1, 78.0, 74.6, 74.5, 74.4, 73.4, 72.6, 72.5, 72.3, 71.8, 69.4, 69.1, 54.9, 21.6. HRMS (ESI) calcd for (M+Na) C₅₄H₅₈O₁₂S: 953.3541, found 953.3541.

Methyl 3-O-(2, 3-di-O-benzyl-5-deoxy-5-methylthio-α-D-xylofuranosyl)-2,4,6-tri-O-benzyl-α-D-mannopyranoside (25): Prepared from 24 (40 mg, 0.04 mmol), 18-crown-6 (10 mg) and sodium thiomethoxide (8 mg, 0.12 mmol) in CH₃CN (I mL) as described for 23, to afford 25 (23 mg, 72%) as a syrup. R_{f}0.38 (4:1, hexanes:EtOAc); [α]_{D}+62.1 (c 0.3, CHCl₃); ¹H NMR (500 MHz, CDCl₃, δ_{H}) 7.46-7.10 (m, 25 H), 5.34 (d, 1 H, J=4.1 Hz), 4.85 (d, 2 H, J=12.0 Hz), 4.76 (d, 2 H, J=12.0 Hz), 4.66 (d, 2 H, J=12.0 Hz), 4.62-4.50 (m, 4 H), 4.45 (d, 1 H, J=12.1 Hz), 4.36 (dd, 1 H, J=6.2, 12.6 Hz), 4.23 (dd, 1 H, J=5.2, 5.2 Hz), 4.12 (dd, 1 H, J=3.1, 9.4 Hz), 4.02 (dd, 1 H, J=9.4, 9.4 Hz), 4.00-3.95 (m, 2 H), 3.82-3.70 (m, 3 H), 3.36 (s, 3 H, OCH₃), 2.75 (dd, 1 H, J=5.6, 13.8 Hz), 2.63 (dd, 1 H, J=7.4, 13.8 Hz, H-5′), 2.08 (s, 3 H, SCH₃); ¹³C NMR (125 MHz, CDCl₃, δ_{C}) 138.9, 138.8, 138.4, 138.0, 137.9, 128.4 (2 C), 128.3 (2 C), 128.2(4) (3 C), 128.2(3), 128.2(1), 127.7, 127.6(8) (2 C), 127.6(4) (3 C), 127.6(3) (2 C), 127.5 (3 C), 127.4, 127.3, 127.2, 127.1 (2 C), 102.2, 99.0, 83.1, 82.0, 79.8, 78.2, 77.7, 74.7, 74.5, 73.4, 72.7, 72.5, 72.4, 71.9, 69.4, 54.8, 34.3, 16.6. HRMS (ESI) calcd for (M+Na) C₄₈H₅₄O₉S: 829.3380, found 829.3383.

Methyl 4-O-(2, 3-di-O-henzyl-5-O-toluenesulfonyl-α-D-xylofuranosyl)-2,3,6-tri-O-benzyl-α-D-mannopyranoside (26): Prepared from thioglycoside 7 (0.76 g, 1.29 mmol), alcohol 11(29) (0.4 g, 0.86 mmol), N-iodosuccinimide (0.35 g, 1.56 mmol) and silver triflate (66 mg, 0.25 mmol) in CH₂Cl₂ (15 mL) as described for 22, to afford 26 (0.71 g, 89%) as a syrup. The product was contaminated with ˜5% of hydrolyzed 7 and thus after characterization by NMR, the disaccharide was used directly in the next step. R_{f}0.28 (4:1, hexanes:EtOAc); ¹H NMR (500 MHz, CDCl₃, δ_{H}) 7.69 (d, 2 H, J=8.3 Hz), 7.40-7.10 (m, 25 H), 7.05-7.00 (m, 2 H), 5.41 (d, 1 H, J=4.3 Hz), 4.83 (s, 1 H), 4.72 (d, 1 H, J=12.4 Hz), 4.65 (d, 1 H, J=12.2 Hz), 4.62-4.53 (m, 3 H), 4.50-4.44 (m, 2 H), 4.38-4.34 (m, 2 H), 4.16 (d, 1 H, J=12.0 Hz), 4.13-3.98 (m, 3 H), 3.94-3.82 (m, 5 H), 3.76 (dd, 1 H, J=4.4, 6.7 Hz), 3.66 (dd, 1 H, J=1.5, 10.5 Hz), 3.55 (dd, 1 H, J=7.3, 10.5 Hz), 3.39 (s, 3 H), 2.36 (s, 3 H); ¹³C NMR (125 MHz, CDCl₃, δ_{C}) 144.6, 138.6, 138.3, 138.1, 137.7, 137.5, 133.0, 129.6 (2 C), 128.4 (2 C), 128.3(4) (2 C), 128.3(0), 128.2(9) (3 C), 128.2, 127.9, 127.8 (2 C), 127.7(4), 127.7(0) (3 C), 127.6(8) (2 C), 127.6 (2 C), 127.5 (2 C), 127.4(3) (2 C), 127.4, 126.8 (2 C), 100.5, 98.4, 82.2, 80.7, 80.1, 74.1, 73.3, 73.1, 72.6, 72.4, 71.9, 71.8, 70.8, 70.5, 69.7, 69.1, 54.8, 21.6. HRMS (ESI) calcd for (M+Na) C₅₄H₅₈O₁₂S: 953.3541, found 953.3540.

Methyl 4-O-(2,3-di-O-benzyl-5-deoxy-5-methylthio-α-D-xylofuranosyl)-2,3,6-tri-O-benzyl-α-D-mannopyranoside (27): Prepared from 26 (0.7 g, 0.75 mmol), 18-crown-6 (60 mg) and sodium thiomethoxide (0.16 g, 2.29 mmol) in CH₃CN (14 mL) as described for 23 to afford 27 (0.46 g, 76%) as a syrup; R_{f}0.3 (4:1, hexanes:EtOAc); [α]_{D}+67.4 (c 0.3, CHCl₃); ¹H NMR (500 MHz, CDCl₃, δ_{H}) 7.40-7.05 (m, 25 H), 5.55 (d, 1 H, J=4.4 Hz), 4.84 (d, 1 H, J=1.7 Hz), 4.74 (d, 1 H, J=12.5 Hz), 4.66 (d, 1 H, J=12.3 Hz), 4.64-4.56 (m, 4 H), 4.54 (d, 1 H, J=11.8 Hz), 4.43 (d, 1 H, J=11.8 Hz), 4.40 (d, 1 H, J=11.8 Hz), 4.22 (d, 1 H, J=12.1 Hz), 4.14 (dd, 1 H, J=9.6, 9.6 Hz), 4.10-4.03 (m, 2 H), 3.97-3.82 (m, 5 H), 3.72 (dd, 1 H, J=7.4, 10.7 Hz), 3.39 (s, 3 H), 2.68 (dd, 1 H, J=4.4, 13.8 Hz), 2.52 (dd, 1 H, J=6.3, 13.8 Hz), 2.06 (s, 3 H); ¹³C NMR (125 MHz, CDCl₃, δ_{C}) 138.7, 138.3, 138.1(4), 138.1, 137.7, 128.4(3), 128.3(8) (2 C), 128.3 (3 C), 128.2(8), 128.2(4) (2 C), 127.8, 127.7 (2 C), 127.6(7) (2 C), 127.6 (3 C), 127.5(8) (2 C), 127.5 (2 C), 127.4, 127.3, 126.8 (2 C), 100.7, 98.5, 82.5, 81.7, 80.3, 77.2, 73.3, 73.2, 72.5, 72.4, 71.8(9), 71.8(8), 71.0, 70.6, 70.1, 54.8, 34.8, 16.6. HRMS (ESI) calcd for (M+Na) C₄₈H₅₄O₉S: 829.3380, found 829.3380.

Methyl 2-O-(2,3-di-O-benzyl-5-O-toluenesulfonyl-α-L-xylofuranosyl)-3-O-benzyl-4,6-O-benzylidene-α-D-mannopyranoside (28): Prepared from thioglycoside 8 (0.12 g, 0.2 mmol), alcohol 9(29) (54 mg, 0.15 mmol), N-iodosuccinimide (0.54 g, 0.24 mmol) and silver triflate (10 mg, 0.04 mmol) in CH₂Cl₂ (3 mL) as described for 22, to afford 28 (89 mg, 73%) as a syrup. R_{f} 0.24 (4:1, hexanes:EtOAc); [α]_{D}−65.6 (c 0.5, CHCl₃); ¹H NMR (500 MHz, CDCl₃, δ_{H}) 7.73 (d, 2 H, J=8.2 Hz), 7.50 (d, 2 H, J=8.2 Hz), 7.45-7.20 (m, 20 H), 5.58 (s, 1 H), 5.58 (s, 1 H), 5.08 (d, 1 H, J=4.0 Hz), 4.70 (s, 1 H), 4.64 (s, 1 H), 4.65-4.54 (m, 3 H), 4.50 (d, 1 H, J=11.0 Hz), 4.46 (d, 1 H, J=11.9 Hz), 4.39 (dd, 1 H, J=5.8, 7.2 Hz), 4.25-4.07 (m, 5 H), 4.03 (dd, 1 H, J=4.2, 5.8 Hz), 3.92 (dd, 1 H, J=3.4, 10.0Hz), 3.80-3.70 (m, 2 H), 3.34 (s, 3 H), 2.39 (s, 3 H); ¹³C NMR (125 MHz, CDCl₃, δ_{C}) 144.5, 138.5, 137.9, 137.8, 137.7, 133.1, 129.7 (2 C), 128.8, 128.4 (2 C), 128.3(5) (2 C), 128.3 (2 C), 128.1(2) (3 C), 128.1, 127.9 (3 C), 127.7 (2 C), 127.5 (2 C), 127.4, 126.1 (2 C), 101.4, 99.1, 97.5, 84.5, 81.4, 78.4, 74.4(4), 74.4, 72.5, 72.1(7), 72.1(5), 71.8, 68.9, 68.8, 64.1, 54.9, 21.6. HRMS (ESI) calcd for (M+Na) C₄₇H₅₀O₁₂S: 861.2915, found 861.2911.

Methyl 2-O-(2 3-di-O-benzyl-5-deoxy-5-methylthio-α-L-xylofuranosyl)-3-O-benzyl-4,6-O-benzylidene-α-D-mannopyranoside (29): Prepared from 28 (44 mg, 0.05 mmol), 18-crown-6 (10 mg) and sodium thiomethoxide (10 mg, 0.18 mmol) in CH₃CN (1 mL) as described for 23, to afford 29 (25 mg, 71%) as a syrup. R_{f}0.33 (4:1, hexanes:EtOAc); [α]_{D}−54.1 (c 0.3, CHCl₃); ¹H NMR (500 MHz, CDCl₃, δ_{H}) 7.55-7.20 (m, 20 H), 5.58 (s, 1 H), 5.17 (d, 1 H, J=4.2 Hz), 4.82 (d, 1 H, J=12.6 Hz), 4.77 (d, 1 H, J=12.6 Hz), 4.73-4.65 (m, 3 H), 4.64-4.52 (m, 3 H), 4.35 (dd, 1 H, J=5.0, 6.6 Hz), 4.28-4.25 (m, 1 H), 4.24 (dd, 1 H, J=4.0, 9.3 Hz), 4.20 (dd, 1 H, J=9.3, 9.3 Hz), 4.10 (dd, 1 H, J=4.7, 4.7 Hz), 3.95 (dd, 1 H, J=3.4, 10.0 Hz), 3.80-3.70 (m, 2 H), 3.35 (s, 3 H), 2.80 (dd, 1 H, J=5.6, 13.8 Hz), 2.65 (dd, 1 H, J=7.6, 13.8 Hz), 2.02 (s, 3 H; ¹³C NMR (125 MHz, CDCl₃, δ_{C}) 138.8, 138.2, 138.0, 137.7, 128.8, 128.4 (2 C), 128.3 (2 C), 128.2 (2 C), 128.1 (2 C), 128.0 (2 C), 127.9, 127.6(1), 127.5(5) (2 C), 127.3(3) (2 C), 127.3, 126.1 (2 C), 101.4, 99.0, 97.5, 84.8, 82.3, 78.6, 76.9, 74.6, 72.4, 72.2, 72.1, 71.9, 68.8, 64.1, 55.0, 34.1, 16.4. HRMS (ESI) calcd for (M+Na) C₄₁H₄₆O₉S: 737.2754, found 737.2756.

Methyl 3-O-(2,3-di-O-benzyl-5-O-toluenesulfonyl-α-L-xylofuranosyl)-2,4,6-tri-O-benzyl-α-D-mannopyranoside (30): Prepared from thioglycoside 8 (170 mg, 0.29 mmol), alcohol 10(29) (93 mg, 0.2 mmol), N-iodosuccinimide (78 mg, 0.35 mmol) and silver triflate (15 mg, 0.06 mmol) in CH₂Cl₂ (4 mL) as described for 22, to afford 30 (150 mg, 82%) as a syrup. The product was contaminated with ˜17% of hydrolyzed 8 and thus after characterization by NMR, the disaccharide was used directly in the next step. R_{f}0.29 (4:1, hexanes:EtOAc); ¹H NMR (500 MHz, CDCl₃, δ_{H}) 7.72 (d, 2 H, J=8.4 Hz), 7.37-7.11 (m, 27 H), 5.14 (d, 1 H, J=4.0 Hz), 4.81 (d, 1 H, J=2.3Hz), 4.80 (d, 1 H, J=11.2 Hz), 4.72-4.58 (m, 4 H), 4.57-4.38 (m, 6 H), 4.35-4.26 (m, 1 H), 4.24-4.14 (m, 3 H), 4.01 (dd, 1 H, J=5.9, 10.6 Hz), 3.95 (dd, 1 H, J=4.0, 5.9 Hz), 3.90 (dd, 1 H, J=8.9, 8.9 Hz), 3.83 (dd, 1H, J=2.5, 2.5 Hz), 3.74-3.72 (m, 2H), 3.37 (s, 3 H), 2.40 (s, 3 H); ¹³C NMR (125 MHz, CDCl₃, δ_{C}) 144.5, 138.6, 138.5, 138.2, 137.8, 137.7, 133.0, 129.7, 129.7, 128.5, 128.4(0) (2 C), 128.3(7) (3 C), 128.3(1), 128.3(0), 128.2(6) (2 C), 128.9(9), 127.9(5), 127.9 (2 C), 127.8 (2 C), 127.7, 127.6(5), 127.6(2) (2 C), 127.6, 127.5(7) (4 C), 127.4(3), 127.4(2), 98.7, 97.3, 83.2, 81.2, 75.7, 74.9, 74.6, 73.3, 72.6, 72.5, 72.5, 72.2, 71.7, 69.4, 68.7, 54.9, 21.6. HRMS (ESI) calcd for (M+Na) C₅₄H₅₈O₁₂S: 953.3541, found 953.3545.

Methyl 3-O-(2, 3-di-O-benzyl-5-deoxy-5-methylthio-α-L-xylofuranosyl)-2,4,6-tri-O-benzyl-α-D-mannopyranoside (31): Prepared from 30 (40 mg, 0.04 mmol), 18-crown-6 (10 mg) and sodium thiomethoxide (10 mg, 0.18 mmol) in CH₃CN (1 mL) as described for 23, to afford 31 (24 mg, 70%) as a syrup. R_{f}0.28 (4:1, hexanes:EtOAc); [α]_{D}−20.5 (c 0.3, CHCl₃); ¹H NMR (500 MHz, CDCl₃, δ_{H}) 7.40-7.20 (m, 25 H), 5.25 (d, 1 H, J=4.1 Hz), 4.89 (d, 1 H, J=11.1 Hz), 4.84 (s, 1 H), 4.74 (d, 1 H, J=4.7 Hz), 4.72 (d, 1 H, J=4.9 Hz), 4.68 (d, 2 H, J=12.3 Hz), 4.63-4.45 (m, 5 H), 4.40 (dd, 1 H, J=6.6, 12.9 Hz), 4.28-4.24 (m, 1 H), 4.24l-4.19 (m, 1 H), 4.04 (dd, 1 H, J=4.1, 4.2 Hz), 3.95 (dd, 1 H, J=8.9, 8.9 Hz), 3.91-3.86 (m, 1 H), 3.85-3.73 (m, 3 H), 3.37 (s, 3 H), 2.78 (dd, 1 H, J=6.4, 13.8 Hz), 2.61 (dd, 1 H, J=6.7, 13.8 Hz), 2.00 (s, 3 H); ¹³C NMR (125 MHz, CDCl₃, δ_{C}) 138.7, 138.5, 138.3, 138.1, 138.0, 128.4, 128.3(1) (3 C), 128.2(8) (4 C), 128.2 (2 C), 127.7(8), 127.7(6) (3 C), 127.7 (2 C), 127.6(8) (3 C), 127.6(6) (3 C), 127.6, 127.5(7), 127.4, 98.7, 97.2, 83.7, 82.3, 77.2, 75.4, 74.8, 74.5, 74.3, 73.3, 72.5, 72.4, 72.2, 71.7, 69.5, 54.9, 33.8,16.3. HRMS (ESI) calcd for (M+Na) C₄₈H₅₄O₉S: 829.3380, found 829.3381.

Methyl 4-O-(2, 3-di-O-benzyl-5-O-toluenesulfonyl-α-L-xylofuranosyl)-2,3,6-tri-O-benzyl-α-D-mannopyranoside (32): Prepared from thioglycoside 8 (0.1 g, 0.17 mmol), alcohol 11(29) (56 mg, 0.12 mmol), N-iodosuccinimide (45 mg, 0.2 mmol) and silver triflate (8 mg, 0.03 mmol) in CH₂Cl₂ (3 mL) as described for 22, to afford 32 (8 mg, 71%) as a syrup. R_{f}0.29 (4:1, hexanes:EtOAc); [α]_{D}−38.6 (c 0.5, CHCl₃); ¹H NMR (500 MHz, CDCl₃, δ_{H}) 7.69 (d, 2 H, J=8.3 Hz), 7.39-7.11 (m, 27 H), 5.05 (d, 1 H, J=4.1 Hz), 4.76 (d, 1 H, J=1.9 Hz), 4.75-4.60 (m, 4 H), 4.55-4.36 (m, 6 H), 4.34-4.20 (m, 3 H), 4.19-4.13 (m, 2 H), 4.10 (dd, 1 H, J=4.1, 10.3 Hz), 3.90 (dd, 1 H, J=5.5, 10.3 Hz), 3.83 (dd, 1 H, J=3.1, 9.0 Hz), 3.80-3.66 (m, 3 H), 3.33 (s, 3 H), 2.36 (s, 3 H); ¹³C NMR (125 MHz, CDCl₃, δ_{C}) 144.4, 138.5, 138.4, 138.3, 137.8, 137.7, 133.0, 129.7, 129.6, 128.5, 128.4 (3 C), 128.3(6), 128.3(2) (2 C), 128.3, 128.2(7), 128.0, 127.9 (2 C), 127.8(6) (2 C), 127.8, 127.7 (4 C), 127.6(4), 127.6(2), 127.5(8) (2 C), 127.5(5), 127.5(3) (2 C), 127.5, 99.4, 99.2, 83.4, 80.8, 78.5, 74.4, 73.9, 73.4, 72.8, 72.7, 72.7, 72.5, 71.9, 71.7, 69.2, 68.7, 54.8, 21.6. HRMS (ESI) calcd for (M+Na) C₅₄H₅₈O₁₂S: 953.3541, found 953.3540.

Methyl 4-O-(2,3-di-O-benzyl-5-deoxy-5-methylthio-α-L-xylofuranosyl)-2,3,6-tri-O-benzyl-α-D-mannopyranoside (33): Prepared from 32 (47 mg, 0.05 mmol), 18-crown-6 (10 mg) and sodium thiomethoxide (10 mg, 0.18 mmol) in CH₃CN (1 mL) as described for 23, to afford 33 (31 mg, 77%) as a syrup. R_{f}0.28 (4:1, hexanes:EtOAc); [α]_{D}−28.8 (c 0.6, CHCl₃); ¹H NMR (500 MHz, CDCl₃, δ_{H}) 7.40-7.20 (m, 25 H), 5.19 (d, 1 H, J=4.1 Hz), 4.784.44 (m, 10 H), 4.41-l4.34 (m, 2 H), 4.23 (dd, 1 H, J=9.2, 9.2 Hz), 4.14 (dd, 1 H, J=6.2, 6.2 Hz), 3.9-3.86 (m, 2 H), 3.82-3.68 (m, 4 H), 3.35 (s, 3 H), 2.68 (dd, 1 H, J=5.9, 13.8 Hz), 2.52 (dd, 1 H, J=6.7, 13.8 Hz), 1.98 (s, 3 H); ¹³C NMR (125 MHz, CDCl₃, δ_{C}) 138.8, 138.5, 138.4, 138.1, 137.9, 128.3(4), 128.3(3) (2 C), 128.2(6) (3 C), 128.2 (2 C), 127.8 (2 C), 127.7(1) (2 C), 127.7 (2 C), 127.6(2) (2 C), 127.5(8), 127.5(5) (2 C), 127.5 (2 C), 127.4(4), 127.4, 99.7, 99.3, 83.9, 81.8, 78.5, 74.8, 73.4, 72.9, 72.7, 72.6(5), 72.6, 72.0, 71.8, 69.5, 54.6, 34.2, 16.4. HRMS (ESI) calcd for (M+Na) C₄₈H₅₄O₉S: 829.3380, found 829.3382.

Methyl 4-O-(5-deoxy-5-sulfoxymethyl-α-D-xylofuranosyl)-α-D-mannopyranoside (34): To a solution of 3 (60 mg, 0.17 mmol) in distilled water (0.3 mL) was added a solution of H₂O₂ (30% aq., 0.019 mL). The reaction mixture was stirred for 9 minutes at room temperature and then lyophilized. The residue was purified by column chromatography on latrobeads (85:15, CH₂Cl₂:CH₃OH) to afford 34 (51 mg, 81%, 1:1 mixture of diastereomers) as a foam. R_{f}0.12 (5.6:1, CH₂Cl₂:CH₃OH); [α]_{D}+160.4 (c 0.3, CH₃OH); ¹H NMR (500 MHz, D₂O, δ_{H}) 5.47 (d, 0.5 H, J=4.5 Hz, H-1′), 5.46 (d, 0.5 H, J=4.4 Hz, H-1′), 4.76 (s, 1H, H-1), 4.65 (ddd, 0.5 H, J=5 5.2, 4.4, 8.5 Hz, H-4′), 4.62 (ddd, 0.5 H, J=5.2, 4.6, 8.5 Hz, H-4′), 4.34 (dd, 1 H, J=5.2, 4.5 Hz, H-3′), 4.23 (dd, 1 H, J=4.5, 4.5 Hz, H-2′), 4.20 (dd, 1 H, J=4.4, 4.5 Hz, H-2′), 3.94-3.85 (m, 3 H, H-2, H-3, H-6), 3.85-3.76 (m, 2 H, H-4, H-6), 3.72-3.66 (m, 1 H, H-5), 3.41 (s, 3 H, OCH₃), 3.29 (dd, 0.5 H, J=4.4, 13.9 Hz, H-5′), 3.15 3.10 (m, 1.0 H, H-5′), 3.09 (dd, 0.5 H, J=8.5, 13.9 Hz, H-5′), 2.81 (s, 1.5 H, S(O)CH₃), 2.80 (s, 1.5 H, S(O)CH₃); ¹³C NMR (125 MHz, D₂O, δ_{C}) 105.6 (1 C, C-1′), 103.7(1 C, C-1), 79.4(0.5 C, C-2′), 79.1 (0.5 C, C-2′), 78.6(0) (0.5 C, C-3′), 78.5(7) (0.5 C, C-3′), 77.0 (0.5 C, C-2), 76.8 (0.5 C, C-2), 76.4 (0.5 C, C-4′), 75.7 (0.5 C, C-4′), 73.8(7) (0.5 C, C-5), 73.8(5) (0.5 C, C-5), 73.5(1) (0.5 C, C-3), 73.49(9) (0.5 C, C-3), 73.0 (1 C, C-4), 63.7(1) (0.5 C, C-6), 63.6(9) (0.5 C, C-6), 57.6 (1 C, OCH₃), 57.2 (0.5 C, C-5′), 55.7 (0.5 C, C-5′), 40.6 (0.5 C, S(O)CH₃), 40.2 (0.5 C, S(O)CH₃). HRMS (ESI) calcd for (M+Na) C₁₃H₂₄O₁OS: 395.0982, found 395.0984.

### Example 2

### Determination of Absolute Stereochemistry and Linkage Position of MTX Residue.

Having synthesized oligosaccharides 1-6, a series of two-dimensional NMR experiments (COSY and HMQC) was carried out on each to fully assign all ¹H and ¹³C resonances for comparison with the data obtained for the MTX residue present in mycobacterial LAM. The chemical shift data of the MTX residue in 1-6 are provided in Table 1, together with the data previously reported for this substituent in M. tuberculosis H37Ra LAM (Treumann et al., supra).

By analyzing this data, it was possible to determine that the MTX residue in the polysaccharide is not of the L-configuration. First, the anomeric hydrogen for this residue in 446 resonates between 5.21 and 5.27 ppm, whereas in the polysaccharide the chemical shift for this hydrogen resonance was reported to be 5.40 ppm, a difference of more than 0.13 ppm. Similarly, the chemical shift of the anomeric carbon residue in 4-6 resonates between 103.0 and 104.6 ppm, which is 0.62.2 ppm lower than that reported for the MTX substituent in the polysaccharide. In contrast, the data for 1-3, which contains an MTX residue with the D-configuration, matches the polysaccharide data better. The MTX anomeric hydrogen resonances in 1-3 are found between 5.30 and 5.41 ppm, differing 0.01-0.1 ppm from the polysaccharide.

TABLE 1
Comparison of NMR chemical shift data for the 5-deoxy-5-methylthio-xylofuranose residue in
1-6 with that found in LAM from M. tuberculosis H37Ra.a

| | ¹H δ (ppm) | | | | | | | ¹³C δ (ppm) | | | | | |
|---|---|---|---|---|---|---|---|---|---|---|---|---|---|
| Compound | H-1 | H-2 | H-3 | H-4 | H-5 | H-5′ | SCH₃ | C-1 | C-2 | C-3 | C-4 | C-5 | SCH₃ |
| 1 | 5.30 | 4.21 | 4.27 | 4.40 | 2.69 | 2.80 | 2.18 | 105.8 | 80.4 | 78.5 | 80.6 | 35.6 | 17.9 |
| 2 | 5.36 | 4.20 | 4.29 | 4.43 | 2.69 | 2.81 | 2.17 | 105.4 | 80.4 | 78.5 | 80.6 | 35.6 | 17.8 |
| 3 | 5.41 | 4.21 | 4.26 | 4.38 | 2.68 | 2.80 | 2.18 | 105.3 | 79.4 | 78.4 | 80.6 | 35.8 | 17.8 |
| 4 | 5.25 | 4.19 | 4.30 | 4.47 | 2.68 | 2.79 | 2.16 | 103.0 | 80.0 | 78.3 | 80.4 | 35.6 | 17.7 |
| 5 | 5.27 | 4.20 | 4.31 | 4.47 | 2.68 | 2.80 | 2.16 | 103.4 | 80.2 | 78.4 | 80.2 | 35.7 | 17.7 |
| 6 | 5.21 | 4.20 | 4.28 | 4.47 | 2.68 | 2.80 | 2.16 | 104.6 | 79.6 | 78.2 | 80.3 | 35.8 | 17.8 |
| Experiment^{b} | 5.40 | 4.21 | 4.26 | 4.38 | 2.68 | 2.80 | 2.21 | 105.2 | 79.4 | 78.3 | 80.5 | 35.8 | 17.4 |

^{a}NMR spectra were recorded in D2O and chemical shifts are referenced to 3-(trimethylsilyl)-propionic acid, sodium salt at 0.0 ppm.
^{b}Taken from Treumann et al., supra.

The chemical shift data for the anomeric carbon compare even better, with these ranging from 105.3-105.8 ppm in 1-3 vs 105.2 in the polysaccharide.

Having established the absolute stereochemistry of the MTX substituent as D, experiments were conducted to determine the position on the mannose residue to which it was linked. Looking first at the ¹H NMR data, the best fit to the polysaccharide is 3, the isomer in which the linkage is α-(1→4). In particular, for the anomeric hydrogen resonance, the chemical shift difference with the polysaccharide is 0.1 ppm (1), 0.04 ppm (2) and 0.01 ppm (3). The same conclusion can be drawn from the ¹³C NMR data. The chemical shift of the anomeric carbon in 3 differed from that reported for the polysaccharide by only 0.1 ppm, as compared to 0.6 and 0.2 ppm for 1 and 2, respectively. However most telling were the differences in the chemical shifts of the MTX C-2 resonances. In 3, the value (79.4 ppm) matched that of the polysaccharide exactly, while in 1 and 2, this resonance was a full ppm more downfield, resonating at 80.4 ppm. Overall, none of the chemical shift data for the polysaccharide differed from that of 3 by more than 0.03 ppm for the ¹H data and 0.4 ppm for the ¹³C NMR data. The is largest differences were seen in the data for the methylthio group (0.03 and 0.4, respectively). When these data are taken out of the comparison, the differences between 3 and the polysaccharide differed by no more than 0.01 ppm for the ¹H data and no more than 0.1 for the ¹³C data. It is not clear why the data for the methylthio group in 3 agrees comparatively poorly with that reported for the polysaccharide, but it is noted that similarly poor agreement was seen in the study establishing the xylo stereochemistry of this substituent.(Turnbull et al., supra) Based on our analysis of these data, it was concluded that the MTX substituent in M. tuberculosis has the D-configuration and is linked α-(1→4) to a mannopyranose residue present in the capping domains.

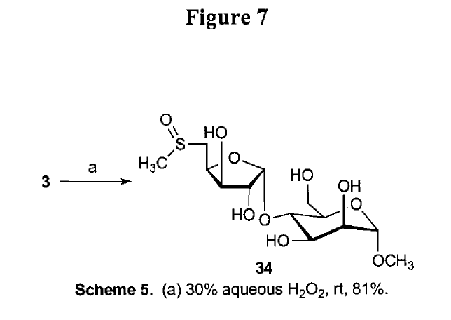

Additional evidence for this assignment was obtained by oxidizing 3 into the corresponding diastereomeric mixture of sulfoxides upon treatment with hydrogen peroxide. As shown in Scheme 5 (FIG. 7), the product was obtained in 81% yield. Comparison of the NMR data for 34 with that of the MSX residue in the polysaccharide (Table 2) showed excellent agreement, thus further bolstering support for the proposed MTX-α-(1→4)-mannopyranose linkage. The ¹H-NMR data for the furanose residue in 34 differed by no more than 0.03 ppm from the polysaccharide, while for the ¹³C-NMR data the chemical shifts were all within 0.4 ppm of those reported. As was the case for 3, the worst agreement was seen for the resonance associated with the methylthio group.

TABLE 2
Comparison of NMR chemical shift data for the diastereomeric 5-deoxy-5-
methylsulfoxy-xylofuranose residues in 34 with those found in LAM
from M. tuberculosis H37Ra.^{a}

| Resonance | 34a | MSP-1^{b} | 34b | MSP-2^{b} |
|---|---|---|---|---|
| H-1 | 5.47 | 5.45 | 5.46 | 5.44 |
| H-2 | 4.23 | 4.22 | 4.20 | 4.20 |
| H-3 | 4.34 | 4.34 | 4.34 | 4.34 |
| H-4 | 4.62 | 4.61 | 4.65 | 4.65 |
| H-5 | 3.12 | 3.12 | 3.29 | 3.28 |
| H-5 | 3.12 | 3.12 | 3.09 | 3.08 |
| S(O)CH₃ | 2.81 | 2.84 | 2.80 | 2.83 |
| C-1 | 105.6 | 105.4 | 105.6 | 105.4 |
| C-2 | 79.1 | 79.3 | 79.4 | 79.4 |
| C-3 | 78.6 | 78.5 | 78.6 | 78.5 |
| C-4 | 75.7 | 75.6 | 76.4 | 76.5 |
| C-5 | 57.2 | 57.1 | 55.7 | 55.6 |
| S(O)CH₃ | 40.6 | 40.2 | 40.2 | 39.9 |

^{a}NMR spectra were recorded in D₂O and chemical shifts are referenced to 3-(trimethylsilyl)-propionic acid, sodium salt at 0.0 ppm.
^{b}Taken from Treumann et al., supra.

As mentioned previously, in addition to being present in M. tuberculosis LAM, the MTX residue has also been found in LAM from M. kansasii (KanLAM) (Gu6rardel et al., supra). However, it was demonstrated that in KanLAM the MTX residue is not attached via the capping motifs of the polysaccharide, but rather to the mannan core. To determine if the linkage position and absolute stereochemistry of the M. kansasii MTX moiety is the same as that in M. tuberculosis, the NMR data for 3 was compared to that obtained for KanLAM (Table 3). As can be seen from the table, there is good agreement between the data for 3 and that for the polysaccharide and thus it is concluded that, like in M. tuberculosis LAM, the MTX residue in KanLAM is also of the D-configuration and is linked α-(1→4) to a mannopyranose residue.

TABLE 3
Comparison of NMR chemical shift
data for the 5-deoxy-5-methylthio-
xylofuranose residue of 3 with that found in
LAM from M. kansasii (KanLAM).^{a}

| Resonance | 3 | KanLAM^{b} |
|---|---|---|
| H-1 | 5.24 | 5.23 |
| H-2 | 3.90 | 3.90 |
| H-3 | 3.98 | 3.99 |
| H-4 | 4.18 | 4.18 |
| H-5 | 2.70 | 2.70 |
| H-5′ | 2.53 | 2.53 |
| S(O)CH₃ | 2.12 | 2.10 |
| C-1 | 104.1 | 103.9 |
| C-2 | 78.4 | 78.0 |
| C-3 | 76.6 | 76.3 |
| C-4 | 80.1 | 79.7 |
| C-5 | 34.5 | 34.4 |
| S(O)CH₃ | 16.8 | 16.5 |

^{a}NMR spectra were recorded in DMSO-d6 and chemical shifts are referenced to the methyl group of the solvent at 2.52 ppm (¹H) or 40.98 ppm (¹³C).
^{b}Taken from Guerardel et al., supra.

### Example 3

### Conformation of the MTX Residue

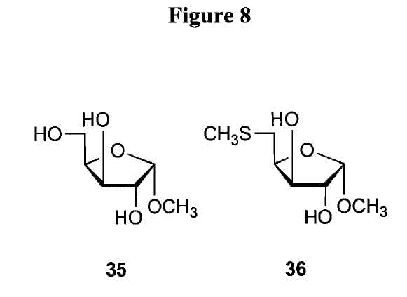

Previous studies of conformational analysis of the methyl α-D-xylofuranoside (35, FIG. 8) showed that it differs from many other furanosides in that it is relatively rigid (Houseknecht et al. (2003) J. Phys. Chem. A. 107:372-378; and Houseknecht et al. (2003) J. Phys. Chem. A. 107:5763-5777). Using NMR spectroscopy and computational chemistry, it was established that the favored ring conformer is an envelope in which C-I is displaced below the plane (E₁), which is very similar to the conformation present in the crystal structure of 35 (Evdokimov et al. (2001) Acta Cryst. B 57:213-220). From analyzing the NMR data for 3 and 34, it became apparent that the coupling constants of the MTX residue were significantly different than those in 35 thus indicating differences in conformation.

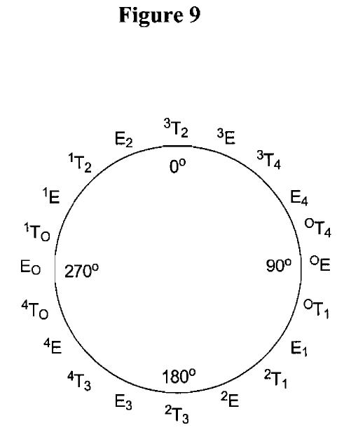

To obtain a more quantitative picture of these conformational differences, PSUEROT calculations (PSEUROT 6.2 (1993) and PSEUROT 6.3 (1999), van Wijk et al., Leiden Institute of Chemistry, Leiden University; de Leeuw and Altona (1983) Comput. Chem. 4:428-437; and Altona (1982) Recl. Trav. Chim. Pays-Bas 101 :413433) were carried out on the MTX rings in 3, the diastereomers of 34, as well as the corresponding methyl glycoside 36 (FIG. 8). The conformation of 36 was evaluated to determine what, if any, role the aglycone plays in the conformational equilibrium of the furanose ring. The PSEUROT approach is a commonly used method for assessing the solution conformation of five-membered rings, and involves the measurement of the three bond ¹H—¹H coupling constants (³J_{HH}) of the ring hydrogens and subsequent analysis of these data. The program assumes a model in which two conformers are present, one in the northern hemisphere of the pseudorotational wheel (Altona and Sundaralingam (1972) Am. Chem. Soc. 94:8205-8212) (FIG. 9), the other in the southern hemisphere. These conformers, termed North (N) or South (S), equilibrate via pseudorotation (Kilpatrick et al. (1947) Am. Chem. Soc. 69:2483-2488; and Pitzer and Donath (1959) J. Am. Chem. Soc. 81:3213-3218).

All calculations were done using PSEUROT 6.3 following modification of the parameters provided for the xylofuranosyl ring. The electronegativities (in D₂0) used were as follows: 1.25 for OH; 1.26 for OR; 0.68 for CH₂OH; 0.62 for CH(OR); 0.0 for H (Altona (1994) Magn. Reson. Chem. 32:670678). For each endocyclic torsion angle, the parameters α and ε were set to 1 and 0, respectively. To translate the exocyclic H,H torsion angles (Φ_{HH}) into the endocyclic torsion angles (νᵢ) that are used to determine the pseudorotational phase angle (P), the program makes use of the relationship: Φ_{HH}=Aνᵢ+B. The values of A and B used were those previously calculated for the methyl α-D-xylofuranoside.(Houseknecht et al. (2002) J. Org. Chem. 67:4647-4651) In all calculations the puckering amplitude, τₘ, was kept constant at 40°, the value found in the crystal structure of 35 (Edokimov et al., supra). These PSUEROT calculations led to the identification of two different solutions, one of which could be eliminated on the basis of the magnitude of the ³J_{C-1-H-4} in 36 (0.5 Hz).

The results of these PSEUROT analyses are provided in Table 4, where they are compared to the populations in the parent structure 35. It is clear that replacement of the C-5 hydroxyl group with the 5-thiomethyl substituent (3, 36) or with the corresponding sulfoxide (34), does alter the conformational equilibrium of the furanose ring. In comparison to 35, the C-5 modified analogs are more flexible, all adopting roughly equimolar mixtures of two conformers, as opposed to an equilibrium in which a single conformer predominates. In addition, this modification alters the conformers present in the equilibrium mixture. Although the identity of the S conformer remains approximately the same, shifting slightly south from El towards ²T₁ (P=124°→P=131-137°), the change in the N conformer is more dramatic, moving from approximately ¹E ( =324°) to ³E (P=13-20°). The origin of this conformational shift is unclear; however, the observation that 3 and 36 have essentially identical conformer distributions rules out the aglycone as a cause of these changes. Beyond that, it is plausible that the conformational shift is driven by eclipsing interactions between OH-3 and the substituent attached to C-5. In the parent structure 35, in which the C-5 substituent is OH, the predominant ring conformer is E₁. The OH-3 and C-5 are nearly perfectly eclipsed in this conformer, but the energetic penalty for this negative interaction is apparently compensated for by the pseudo-axial orientation of the OCH₃ group, which maximizes the anomeric effect. In the minor conformer of 35 (¹E) these groups are also eclipsed. It could be expected that as the size of the C-5 substituent is increased (e.g., changing OH to SCH₃ or S(O)CH₃) these eclipsing interactions become more important, in turn favoring conformations (e.g., ³E) in which C-5 and OH-3 are staggered.

TABLE 4
Results of PSUEROT calculations for 3 and 34-36.^{a,b}

| | Compound | | | | |
|---|---|---|---|---|---|
| | 3 | 34a | 34b | 35^{c} | 36 |
| P_{N} | 14 | 20 | 14 | 324 | 13 |
| % N | 50 | 43 | 45 | 8 | 48 |
| P_{S} | 137 | 135 | 137 | 124 | 131 |
| % S | 50 | 57 | 55 | 92 | 52 |
| RMS^{d} | 0.0 | 0.0 | 0.0 | 0.0 | 0.0 |

^{a}Calculated using a constant Φₘ (Altona-Sundaralingam puckering amplitude) = 40° for all compounds.
^{b}P = Altona-Sundaralingam pseudorotational phase angle.
^{c}Taken from Houseknecht et al. (2003) J. Phys. Chem. A. 107: 5763-5777.
^{d}In Hz.

### Example 4

### Conformation about the C-4C-5 Bond in the MTX Residue

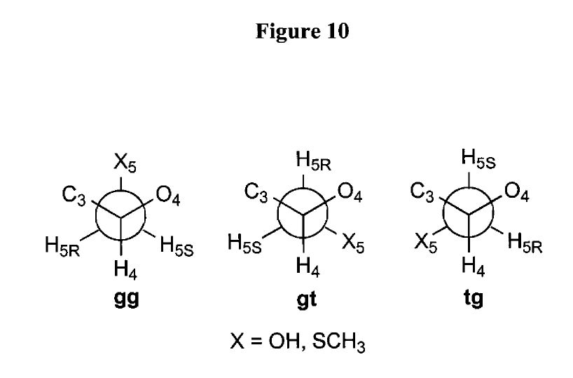

In addition to influencing the conformation of the five membered ring, replacement of the C-5 hydroxyl group with SCH₃ is expected to alter rotamer populations about the C-4C-5 bond (FIG. 10). Thus, the rotamer populations about the C₄-C₅ bond in the furanose residue in 3, 34-36 were determined by analysis of the three bond ¹H—¹H coupling constants between H₄ and H_{5R} (³J_{4,5R}) and H₄ and H_{5S} (³J_{4,5S}) using Equations 1-3, which were derived by taking in account the differences in electronegativities between oxygen and sulfur. In assigning the resonances arising from H_{5R} and H_{5S}, the assumption was made that the chemical shift of H_{5S} is greater than that of H5R, which is the case in the parent glycoside, 35 (Serianni and Barker (1979) Can J. Chem. 57:3160-3167).
2.0 X_{gg}+11.5X_{gt}+3.9 X_{tg}=³J_{4,5R} (1)
3.3 X_{gg}+2.6 X_{gt}+11.5 X_{tg}=³J_{4,5S} (2)
X_{gg}+X_{gt}+X_{tg}=1 (3)

The results of these analyses were compared with the rotamer populations found in 35, which were calculated using the Equations 4-6.
1.1 X_{gg}+10.8 X_{gt}+4.2 X_{tg}=³J_{4,5R} (4)
2.4 X_{gg}+2.9 X_{gt}+10.8 X_{tg}=³J_{4,5S} (5)
X_{gg}+X_{gt}+X_{tg}=1 (6)

The coefficients for Equations 1, 2, 4 and 5 were determined by calculating the limiting ³J_{H,H} for each rotamer using Equation 7.

$${}^{3} {J}_{H , H} = \begin{matrix}14.63 {cos}^{2} θ - 0.78 cos θ + 0.60 + \\ {\sum }_{i} [ 0.34 - 2.31 {cos}^{2} ( {ξ}_{i} θ + 18.4 \vert {χ}_{i} \vert ) ] {χ}_{i}\end{matrix} \qquad ( 7 )$$

For Equation 7, χᵢ is the group electronegativity of the substituents along the coupling pathway and ξᵢ=+1 or −1 as previously defined (Haasnoot (1979) Recl. Trav. Chim. Pays-Bas 98:576-577). The electronegativities used were: 1.25 for OH; 1.26 for OR; 0.70 for SCH₃ and 0.0 for H. The angles θ used in Equation 7 were those of the idealized staggered conformers (60, -60, and 180).

The C-4C-5 rotamer populations for 3, 35 and 36 are presented in Table 5. In the parent structure, 35, the two major rotamers are gg and gt, conformers that are stabilized by a gauche interaction with the ring oxygen (Wolfe (1972) Acc. Chem. Res. 5:102-111). These two rotamers are present in roughly equal amounts, and predominate over the tg conformer, in which the oxygen is trans to the ring oxygen. In the methylthio substituted analogs 3 and 36 this distribution is shifted. In particular, the population of the tg and gt conforrners increase at the expense of the gg rotamer. This change is presumably driven by unfavorable steric interactions between the ring and the comparatively bulky methylthio substituent when adopting the gg conformation. Similarly, the preference for the gt over tg rotamer is likely due to unfavorable steric clashing between the methylthio group and the C-3 hydroxyl group. Previous conformational studies on 4′-thionucleoside derivatives showed a similar increase in tg rotamer when compared to their 4′-oxo counterparts (Cmugelj (2000) J. Chem. Soc., Perkin Trans. 2:255-262). This conformational shift was ascribed, in part, to the preference for 1-alkoxy-2-alkylthio ethane fragments to adopt trans, rather than gauche conformations (Yokoyama and Ohashi (1998) Bull. Chem. Soc. Jpn. 71:1565-1571; and Harada et al. (2002) Chem. Phys. Lett. 362:453-460) and the same stercoelectronic effect may contribute to the differences between rotamer populations in 3 and 36 compared to 35.

TABLE 5
C-4-C-5 rotamer populations
for 3, 35 and 36.^{a}

| | Compound | | |
|---|---|---|---|
| | 3 | 36 | 35 |
| X_{gg}(%) | 14 | 12 | 40 |
| X_{gt}(%) | 63 | 57 | 46 |
| X_{tg}(%) | 24 | 30 | 14 |

^{a}See FIG. 10 for rotamer definitions.

### Example 5

### Effect of 3 and 34 on TNF-α and IL-12p70 Production

Experiments were conducted to determine the role for the MTX residue. Given its location in the capping motif in LAM from M. tuberculosis, it was hypothesized that the residue may function as an immunomodulatory species. Thus, the ability of 3 and 34 to induce or inhibit the production of the TNF-α and IL-12p70 was evaluated using a human monocytic cell line (THP-1). THP-1 cells were re-suspended at a concentration of 5×10⁶ cells/mL in RPMI 1640+10% FCS+1% GPS (200 mM penicillin/streptomycin (Sigma UK)+2 mM L-glutamine (Invitrogen)) and plated into a 48-well plate (500 μL/well). Cells were treated with either 3 or 34 (100 μg and 10 μg/mL) or AraLAM or ManLAM (10 μg/mL) for 24 hours and then stimulated for a further 8 hours with a combination of Staphylococcus aureus Cowan (SAC) (Pansorbin™, Calbiochem, UK) and human IFNγ (1000 U/mL, Preprotech). Following incubation, the supernatants were collected and stored in 200 μL aliquots (−80° C.) and analyzed by ELISA (R&D systems) for IL12p70 and TNF-α production.

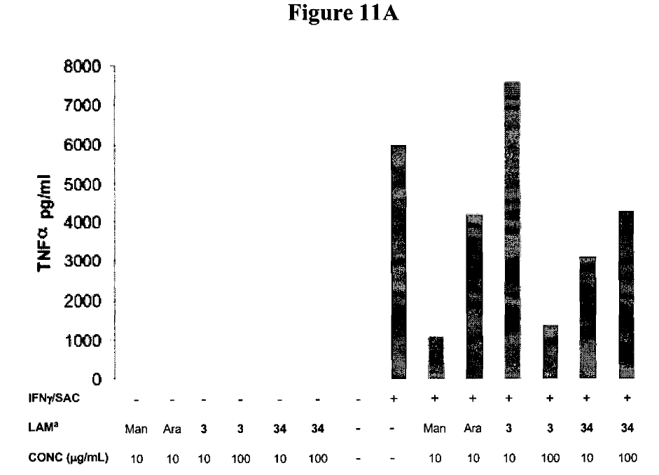

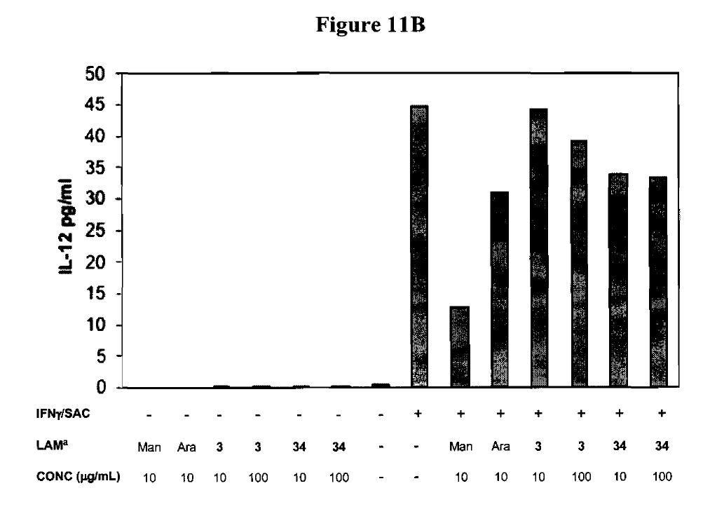

The results of these studies are summarized in FIG. 11. Treatment of THP-1 cells with a preparation of interferon-γ (IFN-γ) and Staphylococcus aureus Cowan strain (IFN-γ/SAC) led to a strong production of both TNF-α (FIG. 11A) and IL-12p70 (FIG. 11B). Neither 3 nor 34, when tested at concentrations of 10 or 100 μg/mL, significantly induced the production of these two cytokines. As a comparison, both ManLAM and AraLAM were tested at 10 μg/mL, and also did not lead to TNF-α or IL-12p70 induction. When 3 and 34 were tested as inhibitors of the cytokine response induced by IFN-γ/SAC, modest levels of inhibition were observed. For TNF-α (FIG. 11A), 3 at a concentration of 100 μg/mL led to a level of inhibition comparable with ManLAM at 10 μg/mL, whereas 34 (at 10 μg/mL) was less effective and comparable to AraLAM at 10 μg/mL. These compounds were poorer inhibitors of IL-12p70, with both 3 and 34 exerting only a very modest effect at either 10 or 100 μg/mL.

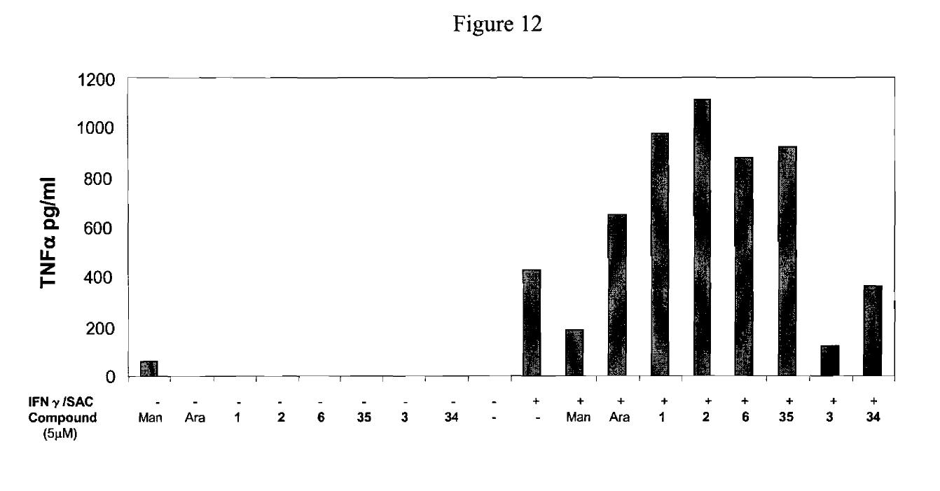

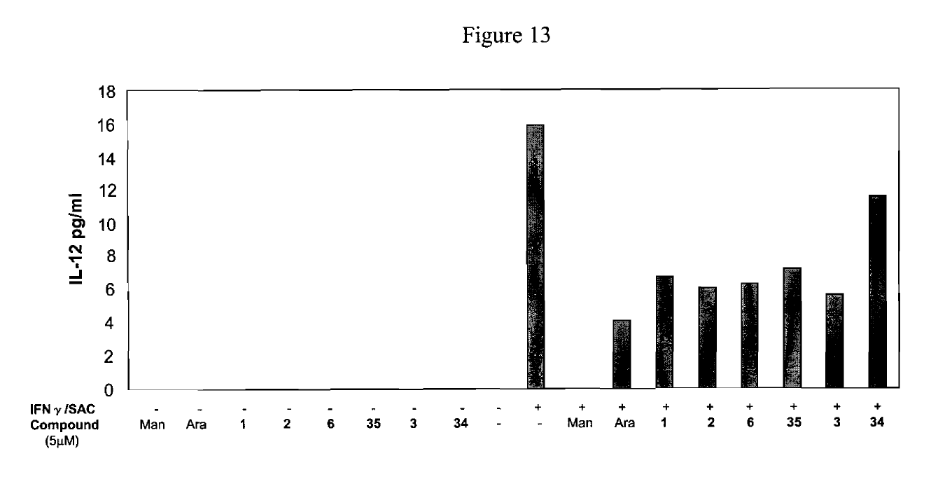

Because of the significant molecular weight differences between 3, 34 and the two polysaccharides, assays also were carried out in which the concentration of these compounds was kept constant (FIGS. 12 and 13). A concentration of 5 VLM was used in these assays, which is the approximate molarity of a 100 μg/mL solution of ManLAM (mw 17,400). For the TNF-α assays (FIG. 12), the trends were the same as those shown in FIG. 11A, i.e., a 5 μM concentration of 3 inhibited TNF-α production to a similar degree as a 5 μM concentration of ManLAM. In addition, 34 was a weaker inhibitor than 3. The results with IL-12p70 (FIG. 13) was also similar to those shown in FIG. 11B, neither 3 or 34 at 5 μM inhibited the production of the cytokine to the degree of the same concentration of ManLAM. For IL-12p70, 3 had a similar activity as AraLAM, whereas 34 was less active.

Finally, compounds 1, 2, 6 and 35 at 5 μM were tested as controls in both assays. In the case of TNF-α, none of these compounds inhibited cytokine induction (FIG. 12). Indeed, each appeared to induce production of TNF-α to varying degrees. For IL-12p70, all four of these compounds also inhibited induction, but to a degree intermediate between 3 and 34. These results suggest that the inhibition of TNF-α by 3 and 34 is specific to the structures of the molecules, while for IL-12p70 the effect is non-specific.

In summary, through the combined use of chemical synthesis and NMR spectroscopy, it has been established that the 5-deoxy-5-methylthio-xylofuranose (MTX) and 5-deoxy-5-methylsulfoxy-xylofuranose (MSX) residues present in the LAM of M. tuberculosis and M. kansasii are of the D-configuration and are linked α-(1→4) to a mannopyranose residue in the glycan. Conformational analysis of these residues indicated differences in both ring conformation and rotamer populations about the C-4-C-5 bond, as compared to the parent compound, methyl α-D-xylofuranoside (35). Two of the synthesized disaccharides, 3 and 34, when tested in assays of cytokine induction did not lead to production of TNF-α or IL12-p70; however, both showed modest inhibitory properties when these cytokines were induced using SAC/IFN-γ. These latter observations suggest that this motif may play a role in the immune response arising from mycobacterial infection. Thus, this class of compounds are useful in modulating immune responses, treating inflammatory disorders or conditions, treating autoimmune disorders or conditions, treating rheumatoid arthritis and modulating levels of lymphokines and cytokines (e.g., TNF-α and IL-12).

Various in vitro and in vivo assays known in the art are used to demonstrate the immune modulating capacity of MTX/MSX residues. In addition, various animal models and disease animal models known in the art are utilized to demonstrate efficacy of these oligosaccharides as treatment modalities. These include, without limitation, animal models for RA, SLE, lupus nephritis, MS, PD, Crohn's disease, psoriasis, autoimmune glomerulonephritis, atherosclerosis, ankylosing spondylitis, graft rejection and transplantation.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A saccharide compound comprising a 5-deoxy-5-methylthio-xylofuranose (MTX) or 5-deoxy-5-methylsulfoxy-xylofuranose (MSX) moiety, or a derivative thereof

2. The compound of claim 1, wherein said MTX or MSX moiety or derivative thereof is in the D-configuration.

3. The compound of claim 1, wherein said saccharide is a monosaccharide or a disaccharide.

4. The compound of claim 1, wherein said compound is selected from Formulas 1-6:

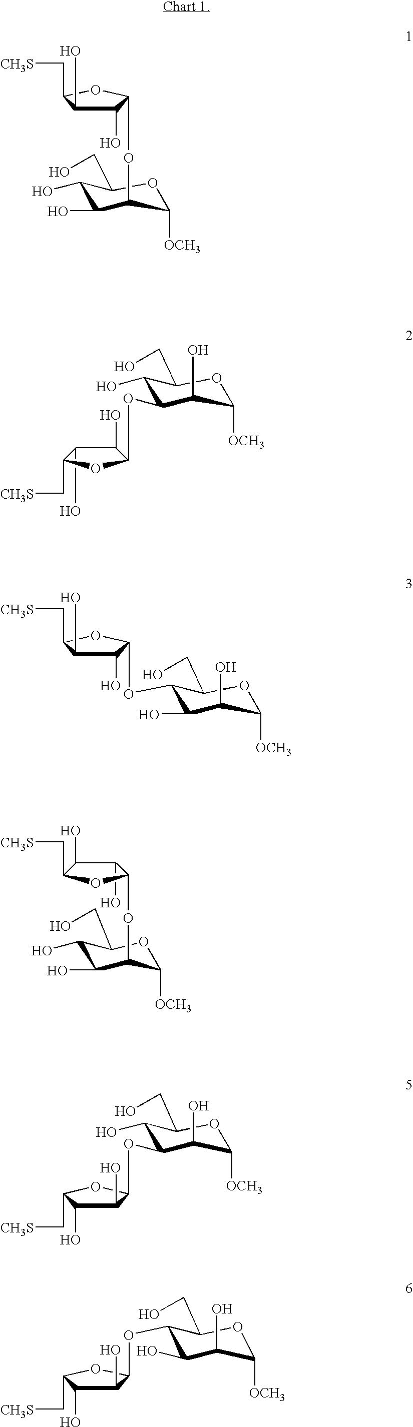

5. The compound of claim 1, wherein said compound is the oxidized form of any one of compounds 1-6.

6. The compound of claim 5, wherein said compound is Formula 34:

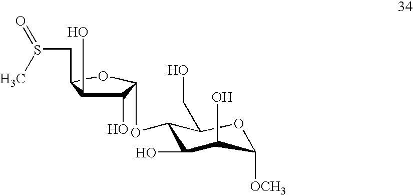

7. A compound comprising a derivative of a MTX or MSX moiety, wherein said compound is a tosylated thioglycoside.

8. The compound of claim 7, wherein said derivative of a MTX or MSX moiety is selected from Formula 7 and Formula 8:

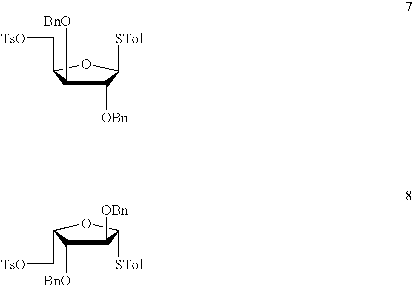

9. A composition comprising the saccharide compound of claim 1.

10. The composition of claim 9, further comprising a pharmaceutically acceptable carrier.

11. A method for modulating an immune response in a mammal, comprising administering to a mammal in need thereof the saccharide compound of claim 1 or the composition of claim 9.

12. A method for treating an inflammatory disorder in a mammal, comprising administering to a mammal diagnosed with an inflammatory disorder the saccharide compound of claim 1 or the composition of claim 9.

13. A method for treating an autoimmune disorder in a mammal, comprising administering to a mammal diagnosed with an autoimmune disorder the saccharide compound of claim 1 or the composition of claim 9.

14. The method of claim 13, wherein said autoimmune disorder is rheumatoid arthritis.

15. A method for modulating cytokine, lymphokine, or chemokine levels in a mammal, comprising administering to said mammal the saccharide compound of claim 1 or the composition of claim 9.

16. The method of claim 15, wherein said cytokine, lymphokine, or chemokine is TNF-α.

17. The method of claim 15, wherein said cytokine, lymphokine, or chemokine is IL-12.
